(19) United States
(12) Patent Application Publication
(10) Pub. No.: US 2013/0288364 A1
(43) Pub. Date: Oct. 31, 2013

(54) ENGINEERED KINETOCHORES AND USES THEREOF

(71) Applicants: Iain Cheeseman, Brookline, MA (US); Karen Gascoigne, San Francisco, CA (US)

(72) Inventors: Iain Cheeseman, Brookline, MA (US); Karen Gascoigne, San Francisco, CA (US)

(73) Assignee: Whitehead Institute for Biomedical Research, Cambridge, MA (US)

(21) Appl. No.: 13/882,028

(22) PCT Filed: Oct. 28, 2011
(86) PCT No.: PCT/US11/58425

§ 371 Date: Jul. 8, 2013

Related U.S. Application Data
(60) Provisional application No. 61/407,824, filed on Oct. 28, 2010.

(51) Int. Cl. C07K 14/47

(52) U.S. Cl. CPC C07K 14/47
USPC 435/349; 530/350; 536/23.5; 435/320.1; 530/358; 435/325

(57) ABSTRACT

The invention provides modified DNA-binding kinetochore polypeptides, wherein the modified DNA binding kinetochore polypeptides comprise a heterologous DNA binding domain. The invention further provides engineered kinetochores containing the modified kinetochore polypeptides. Further provided are artificial chromosomes containing an engineered kinetochore. Cells containing an artificial chromosome containing an engineered kinetochore are also provided. Methods for producing and methods of using the engineered kinetochore, artificial chromosome, and cells are also provided.

### CROSS-REFERENCE TO RELATED APPLICATION

This application claims the benefit of U.S. Provisional Application No. 61/407,824, filed Oct. 28, 2010. The entire teachings of the above application are incorporated herein by reference.

### GOVERNMENT SUPPORT

This invention was made with government support under GM088313 awarded by the National Institute of General Medical Sciences. The government has certain rights in the invention.

### BACKGROUND OF THE INVENTION

Accurate chromosome segregation is essential for proper transmission of genetic material during cell division. The kinetochore is a proteinaceous macro-molecular structure that forms at the centromere of each chromosome and connects the chromosome to microtubules of the mitotic spindle during mitosis, thereby facilitating chromosome segregation. For accurate chromosome segregation to occur, it is important that only one functional kinetochore assembles on each sister chromatid. However, the way in which this site of assembly is specified has been unclear. A better understanding of the mechanisms that direct kinetochore assembly would be of considerable scientific and practical interest.

### SUMMARY OF THE INVENTION

The invention relates at least in part to the discovery that two DNA-binding kinetochore components, CENP-C and the CENP-T/W complex, function downstream of CENP-A to direct kinetochore formation. Replacing the DNA-binding regions of CENP-C and CENP-T with alternate chromosome targeting domains recruits these proteins to ectopic loci and results in assembly of additional kinetochore proteins. These ectopic kinetochore foci are functional based on the presence of representative kinetochore components including the microtubule binding KMN network, and the segregation behavior of chromosomes which contain these foci.

In some aspects, the invention provides modified kinetochore polypeptides and nucleic acids that encode them. In some aspects, the invention provides engineered kinetochores comprising one or more modified kinetochore polypeptides. In some aspects, the invention provides a chromosome that has a site for assembly of an engineered kinetochore. In some aspects, the invention provides a chromosome having an engineered kinetochore assembled thereon. Cells and cell lines comprising one or more inventive polypeptides, nucleic acids, engineered kinetochores, and/or artificial chromosomes are also provided. Methods of using an engineered kinetochore, artificial chromosome, or cell containing an artificial chromosome of the invention, are also provided.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/ and in Online Mendelian Inheritance in Animals (OMIA), a database of genes, inherited disorders and traits in animal species (other than human and mouse), at http://omia.angis.org.au/contact.shtml. Non-limiting information regarding kinetochores and kinetochore components may be found in, e.g., DeWulf, P., et al. (eds.). The Kinetochore, Springer, 2009. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. CENP-A, the CENP-T/W complex and CENP-C are essential for kinetochore assembly. A) Images of HeLa cells 24 hours after transient expression of GFP-CENP-A. Images show varying levels of expression, with localization to chromosome arms at high levels. B) Representative images of kinetochore components not mis-localized by CENP-A over-expression. CENP-T and Hec1 are visualized by immunofluorescence, CENP-H is visualized using a HeLa cell line expressing GFP^{LAP}-CENP-H, C) Images show mis-localization of kinetochore components to chromosome arms in the presence of ectopic CENP-A, or centromeric localization in the presence of GFP-H2B (control). CENP-C was visualized by immunofluorescence, or by co-over-expression of mCherry-CENP-C. CENP-N or Mis18 were visualized using HeLa cell lines stably expressing GFP^{LAP}-CENP-N and Mis18. D) Graph showing quantification of mitotic index 48 h after depletion of CENP-C, CENP-T, or CENP-C & CENP-T by RNAi. N=100, error bars show standard error of the mean (s.e.m). E) Quantification of kinetochore intensity after 48 h following RNAi depletion of CENP-C, CENP-T, or CENP-C & CENP-T. N=>50 kinetochores from at least 5 cells, error bars show s.e.m. F) Representative immunofluorescence images of HeLa cells 48 hours after RNAi depletion of the indicated proteins. Merge inserts show ACA in red, CENP-H in green. All scale bars show 5 μm.

FIG. 2. CENP-T N and C termini are required for kinetochore assembly and DNA binding, respectively. A) Coomassie-stained gel showing recombinant 6×His-CENP-T and CENP-W co-purified from bacteria. Gel shows co-purification of wild-type CENP-T/W complex, CENP-T-ΔN/W, CENP-T/Wₘᵤₜ, CENP-T-ΔN/Wₘᵤₜ. B) EMSA showing retardation of a biotinylated 20 bp alpha-satellite DNA probe by incubation with the indicated CENP-T/W complexes. Retardation is reduced in the presence of CENP-Wₘᵤₜ compared to wild type CENP-W. C) Graph showing quantification of mitotic index 48 h after RNAi depletion of CENP-W in HeLa cells or HeLa cell lines expressing RNAi resistant CENP-W or CENP-Wₘᵤₜ. N=100 cells, error bars show s.e.m. D) Graph showing quantification of the mitotic index 48 h after RNAi depletion of CENP-T in HeLa cells, or HeLa cell lines expressing RNAi resistant CENP-T or CENP-TAN. N=100 cells, error bars show s.e.m. E) Representative immunofluorescence images of chicken DT40 cells after depletion of endogenous CENP-T and expression of the indicated protein fragments. Scale bar, 10 μm. F) Quantification of kinetochore intensity after depletion of CENP-T and expression of the indicated proteins. N=200, +/−SD. G) Graph showing viability of CENP-T conditional-depletion in DT40 cells expressing the indicated proteins after addition of tetracycline. H) Graph showing the elution profile (OD₂₈₀) of recombinant CENP-T/W complex and CENP-T-ΔN/W on a Superose 6 size exclusion column. Arrows indicate the migration of standards with known Stokes radii: Thyroglobulin (85 Å), Aldolase (48.1 Å) and RNase A (16.4 Å). I) Immunofluorescence images showing levels of Hec1/Ndc80 48 h after RNAi depletion of CENP-T in HeLa cells or HeLa cell lines expressing RNAi resistant GFP-CENP-T or GFP-CENP-T-ΔN. Scale bar, 5 μm.

FIG. 3. Targeting of CENP-C and CENP-T to chromatin by fusion with histone H2B causes mis-localization of the KMN network and interferes with chromosome segregation. A) Images showing centromeric localization of DNA binding kinetochore proteins in cells transiently expressing GFP-CENP-T-ΔC-H2B or GFP-CENP-C-ΔC-H2B. CENP-A, CENP-T and CENP-C are visualized by immunofluorescence. B) Images showing mis-localization of KMN network proteins to chromosome arms in cells transiently expressing GFP-CENP-T-ΔC-H2B or GFP-CENP-C-ΔC-H2B or cells co-expressing mCherry-CENP-T-ΔC-H2B (not shown) and GFP-CENP-C-ΔC-H2B. Dsn1, KNL1 and Ndc80/Hec1 are visualized by immunofluorescence. C) Left, images showing centromeric localization of the indicated kinetochore proteins in cells expressing GFP-H2B as a control protein. Right, images showing mis-localization of the indicated proteins to chromosome arms in cells transiently co-expressing mCherry-CENP-T-ΔC-H2B (not shown) and GFP-CENP-C-ΔC-H2D. Zwint, Nup133 and INCENP are visualized by immunofluorescence. Scale bar shows 5 μm. D) Graph showing quantification of stages of mitosis in fixed cells expressing GFP-CENP-T-ΔC-H2B and GFP-CENP-C-ΔC-H2B, individually or in combination. N=100 cells per condition. E) Left. graph showing quantification of the duration of mitosis from Nuclear Envelope Breakdown (NEBD) to chromatin decondensation in cells expressing GFP-H2B, GFP-CENP-T-ΔC-H2B, GFP-CENP-C-ΔC-H2B or co-expressing mCherry-CENP-T-ΔC-H2B (not shown) and GFP-CENP-C-ΔC-H2B. N>50 cells per condition. Bar indicates the mean. Middle, selected images from time-lapse movies of HeLa cells expressing mCherry-CENP-T-ΔC-H2B (not shown) and GFP-CENP-C-ΔC-H2B. Top panel shows mitotic arrest, bottom panel shows accelerated mitosis. Time is indicated in minutes after NEBD. Right, graph showing quantification of the mitotic exit phenotype from live cell imaging for cells expressing GFP-H2B, GFP-CENP-T-ΔC-H2B, GFP-CENP-C-ΔC-H2B or co-expressing mCherry-CENP-T-ΔC-H2B (not shown) and GFP-CENP-C-ΔC-H2B. N>50 cells. Scale bars show 5 μm.

FIG. 4. The CENP-T/W complex and CENP-C interact directly with KMN network components in vitro. A) The CENP-T/W complex can bind directly to CENP-K, GST-Ndc8^{0Bonsai}, and the Mis12 complex in vitro. Left, Coomassie-stained gel showing 6×His-CENP-T/W complex immobilized on Ni-NTA agarose resin, alone or in the presence of GST or GST-CENP-K. The first lane shows 1% input. Middle, Coomassie-stained gel showing GST-Ndc8^{0Bonsai} or GST immobilized on glutathione agarose in the presence of 6×His-CENP-T/W complex. The first lane shows 1% input. Right, Coomassie-stained gel shows GST-CENP-^{T} or GST immobilized on glutathione agarose in the presence of 6×His tagged Mis12 complex. The first lane shows 1% input. The lower panel shows a western blot of the same proteins, probed with anti-DSN1 antibody. B) CENP-C can bind directly to the Mis12 complex in vitro. Western blot showing the elution profile for GST-CENP-C AC (amino acids 1-235) alone (top panel) or shifted in the presence of pre-assembled Mis12/Ndc8^{0Bonsai} complex (bottom panels). The first lane shows 1% input. Blots are probed with antibodies against CENP-C and Dsn1. C) Data from the mass spectrometric analysis of one step immunoprecipitation purifications of either endogenous CENP-C, or LA^{PGFP}-Mis12. D) More extensive data from mass spectrometric analysis of one step immunoprecipitation purifications of either endogenous CENP-C, or LA^{PGFP}-Mis12. Diverse samples prepared using identical conditions serve as negative controls for these purifications.

FIG. 5. Targeting of CENP-C and CENP-T to a specific chromatin locus via a LacO/LacI system causes mis-localization of the KMN network. A) Images show representative metaphase spread chromosomes from U2OS-LacO cells. Bottom images shows chromosome 1 with GFP-T-ΔC-LacI present at the LacO array. Hec1 is visualized by immunofluorescence. B) Images showing centromeric localization of DNA binding kinetochore proteins in cells transiently expressing GFP-CENP-T-ΔC-LacI or GFP-CENP-C-ΔC-LacI. CENP-A, CENP-T and CENP-C are visualized by immunofluorescence after 15 h Nocodazole treatment. Inserts show a merge of kinetochore protein in red, and LacI foci in green. C) Images showing co-localization of KMN network proteins with LacI foci in cells transiently expressing GFP-CENP-T-ΔC-LacI or GFP-CENP-C-ΔC-LacI or co-expressing mCherry-CENP-T-ΔC-LacI (not shown) and GFP-CENP-C-ΔC-LacI, but not a GFP-LacI control protein. Dsn1, KNL1 and Ndc80/Hec1 are visualized by immunofluorescence. Inserts show a merge of kinetochore protein in red, and LacI foci in green. D) Images showing interphase co-localization of Dsn1, but not Ndc80/Hec1, with LacI foci in cells expressing GFP-CENP-C-ΔC-LacI or co-expressing mCherry-CENP-T-ΔC-LacI and GFP-CENP-C-ΔC-LacI, but not in cells expressing GFP-CENP-T-ΔC-LacI alone. Dsn1 and Ndc80/Hec1 are visualized by immunofluorescence. Inserts show a merge of kinetochore protein in red, and LacI foci in green. E) Images showing co-localization of Ndc80/Hec1, but not Dsn1, with GFP-CENP-T-ΔC-mito in the cytosol. Dsn1 and Ndc80/Hec1 are visualized by immunofluorescence. Scale bars show 5 μm. F) Quantitation of the relative fluorescence of the indicated kinetochore proteins at endogenous kinetochores versus ectopic foci in cells expressing LacI fusion proteins. Quantification was conducted after 15 h nocodazole treatments, >10 cells/condition, 20 kinetochores/cell, +/−SEM. Data is shown normalized to the foci/kinetochore ratio of the fusion protein. G) Schematic representation of CENP-T and CENP-C. The N-terminal regions used in fusion protein experiments are indicated in yellow (amino acids 1-242 for CENP-T and 1-235 for CENP-C).

FIG. 6. Induced CENP-T and CENP-C foci recruit outer kinetochore proteins and function in chromosome segregation. A) Top, images showing centromeric localization of the indicated kinetochore proteins in cells expressing GFP-LacI control protein. Bottom, images showing co-localization of outer kinetochore proteins with LacI foci in cells transiently co-expressing mCherry-CENP-T-ΔC-LacI (not shown) and GFP-CENP-C-ΔC-LacI. Zwint, Aurora B, Ska1, Nup133, and phospho-Dsn1 are visualized by immunofluorescence after 15 h Nocodazole treatment. Inserts show a merge of kinetochore protein in red, and LacI foci in green. B) Representative images of cells co-expressing mCherry-CENP-T-ΔC-LacI (not shown) and GFP-CENP-C-ΔC-LacI, or GFP-LacI, untreated or treated with 3.3 M Nocodazole. Images show deformation of the circular LacI foci in the presence of microtubule. Microtubules are visualized by immunofluorescence. The mean length/width ratio for the foci is shown below each image. N=10 cells. C) Graph showing the percentage of cells which show more than one LacI foci 72 hours after expression of GFP-LacI or GFP-CENP-T-ΔC-LacI. N=200 cells D) Graph showing quantification of LacI foci segregation at anaphase in live cells expressing GFP-LacI, GFP-CENP-T-ΔC-LacI, or GFP-CENP-C-ΔC-LacI. E) Selected images from time-lapse movies of U2OS-LacO cells expressing mCherry-histone-H2B to visualize chromatin, and GFP-CENP-T-ΔC-lacI or GFP-LacI control protein, showing segregation of LacI foci at anaphase. Scale bars show 5 μm.

FIG. 7. Induced CENP-T and CENP-C foci recruit regulatory and outer kinetochore proteins. A) Left, immunofluorescence images showing co-localization of kinetochore proteins with LacI foci in cells transiently co-expressing mCherry-CENP-T-ΔC-LacI (not shown) and GFP-CENP-C-ΔC-LacI after 15 h Nocodazole treatment. Inserts show a merge of kinetochore protein (red) and LacI foci (green). Right, co-localization of GFP-CENP-N in cells expressing mCherry-CENP-T-ΔC-LacI or mCherry-CENP-C-ΔC-LacI. B) Quantification of the percentage of focus-containing cells with the indicated kinetochore protein at the ectopic site. N>20. C) Quantification of the relative fluorescence of the indicated kinetochore proteins at kinetochores versus ectopic foci, in cells expressing LacI fusion proteins. Quantification was done after 15 h nocodazole treatment, N>10 cells/condition, 20 kinetochores/cell, +/−SEM. Data are shown normalized to the foci/kinetochore ratio of the fusion protein. D) Top, Immunofluorescence images showing co-localization of Mad2 with LacI foci in cells transiently co-expressing mCherry-CENP-T-ΔC-LacI (not shown) and GFP-CENP-C-ΔC-LacI after 15 h Nocodazole treatment, or 1 h treatment with MG132. Bottom, Quantification of Mad2 fluorescence at ectopic foci after 15 h nocodazole treatment or 1 h treatment with MG132, N≧10 cells/condition, 20 kinetochores/cell, +/−SEM. Asterisk indicates significant difference as determined by Mann Whitney U test P<0.005. Data are shown normalized to GFP fluorescence at ectopic foci. E) Immunofluorescence images showing co-localization of SAC proteins with LacI foci in chicken DT40 cells containing a LacO array, and expressing GFP-CENP-T-ΔC-LacI. Scale bar, 10 μm.

FIG. 8. Induced CENP-T and CENP-C foci interact with microtubules and function in chromosome segregation. A) Representative immunofluorescence images of cells expressing GFP-LacI, or co-expressing RNAi resistant mCherry-CENP-T-ΔC-LacI (not shown) and GFP-CENP-C-ΔC-LacI. Microtubules are shown in red. Cells were treated with 2 μM ZM447439 or 3.3 M Nocodazole, or 48 h CENP-C and CENP-T RNAi as indicated. In all cases, cells were cold treated for 20 min prior to fixation to visualize stable kinetochore microtubule fibers. Arrows indicate the foci. The mean length/width ratio for the foci is shown below the indicated images. N>5 cells. B) Electron micrographs of an ectopic CENP-T-LacI foci showing the presence of microtubule attachments (red arrow). Dark spots indicate immuno-labeling with anti-Hec1 antibody. This ectopic CENP-T-LacI foci was defined by the size and extensive anti-Hec1 labeling of this structure relative to an endogenous kinetochore. Direct correlative light-EM is shown to analyze ectopic kinetochore structure in FIG. 9. C) Graph showing the percentage of cells with more than one LacI foci 72 h after expression of GFP-LacI or GFP-CENP-T-ΔC-LacI. N=200 cells D) Graph showing quantification of LacI foci segregation in live cells expressing GFP-LacI, GFP-CENP-T-ΔC-LacI, or GFP-CENP-C-ΔC-LacI. E) Selected images from time-lapse movies of U2OS-LacO cells expressing mCherry-histone-H2B to visualize chromatin, and GFP-CENP-T-ΔC-lacI or GFP-LacI showing segregation of LacI foci at anaphase. Time in shown in minutes after NEBD. Scale bars, 5 μm. F). Centromere replacement assay in chicken DT40 cells (see FIG. 9B-9C for a schematic of this strategy). Left and middle panels; representative images of DT40 cells with GFP-LacI fusion protein localized to a LacO array on the Z chromosome (arrows), after Cre recombinase mediated excision of the centromeric region of the same chromosome. Left, representative images of the effected chromosome lagging at anaphase. Middle, representative images of correct segregation of the Z chromosome. Right panel; graph showing the percentage of GFP foci containing cells with lagging or equally dividing Z chromosomes 18 h after addition of tamoxifen to induce excision of the endogenous centromere cells. N=78 cells per condition.

FIG. 9. Ectopic foci interact with microtubules. A) Chromosome spreads observed by electron microscopy (EM). Left panels shown fluorescence microscope images of a control chromosome, and corresponding EM image of the same chromosome. Red signals were visualized by anti-CENP-T antibody. Right panels show fluorescence microscope images of a CENP-T-LacI foci-containing chromosome and corresponding EM image. A primary constriction-like structure was observed at the position where CENP-T accumulated. The length of the structure is ˜1.5 μm relative to ˜0.3 μm for an endogenous kinetochore. Scale bars: IF, 10 μm; EM, 1 μm. B) CENP-T was targeted to the cytoplasmic surface of the mitochondrial outer membrane using a “mito” tag (Bear et al., 2000). Representative immunofluorescence images showing co-localization of Ndc80/Hec1, with GFP-CENP-T-ΔC-mito, but not with GFP-CENP-T-ΔC-mito with 5 CDK phosphorylation sites mutated to alanine (S-A). Scale bar, 5 μm. (B and C) Schematic representation of strategies used to remove the centromeric region of the Z chromosome in chicken DT40 cells, and to generate an ectopic LacO/LacI foci on the same chromosome. C) Southern blot analysis of genomic DNA from DT40 cells expressing GFP-LacI or GFP-CENP-T-ΔC-LacI showing similar levels of excision in each case. DNA was digested with Afl II and probed for the bleomycin cassette. The presence of a 17.3 kb fragment indicates excision of the centromeric region.

FIG. 10. Model depicting interactions at endogenous and ectopic kinetochores in certain embodiments. Orange circles containing the letter “P” represent phosphorylation.

### DETAILED DESCRIPTION OF CERTAIN EMBODIMENTS OF THE INVENTION

### I. Definitions

The term “antibody” encompasses immunoglobulins and derivatives thereof containing an immunoglobulin domain capable of binding to an antigen. An antibody can originate from a mammalian or avian species, e.g., human, rodent (e.g., mouse, rabbit), goat, chicken, etc., or can be generated ex vivo using a technique such as phage display. Antibodies include members of the various immunoglobulin classes, e.g., IgG, IgM, IgA, IgD, IgE, or subclasses thereof such as IgG1, IgG2, etc. In various embodiments of the invention “antibody” refers to an antibody fragment or molecule such as an Fab′, F(ab′)2, scFv (single-chain variable) that retains an antigen binding site and encompasses recombinant molecules comprising one or more variable domains (VH or VL). An antibody can be monovalent, bivalent or multivalent in various embodiments. The antibody may be a chimeric or “humanized” antibody. An antibody may be polyclonal or monoclonal, though monoclonal antibodies may be preferred. In some aspects, an antibody is an intrabody, which may be expressed intracellularly.

An “effective amount” or “effective dose” of a compound or other agent (or composition containing such compound or agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular compound, agent, or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or the desired effect may be achieved by use of multiple doses. An effective amount of a composition may be an amount sufficient to reduce the severity of or prevent one or more symptoms or signs of a disorder.

“Identity” or “percent identity” is a measure of the extent to which the sequence of two or more nucleic acids or polypeptides is the same. The percent identity between a sequence of interest A and a second sequence B may be computed by aligning the sequences, allowing the introduction of gaps to maximize identity, determining the number of residues (nucleotides or amino acids) that are opposite an identical residue, dividing by the minimum of TG_{A} and TG_{B} (here TG_{A} and TG_{B} are the sum of the number of residues and internal gap positions in sequences A and B in the alignment), and multiplying by 100. When computing the number of identical residues needed to achieve a particular percent identity, fractions are to be rounded to the nearest whole number. Sequences can be aligned with the use of a variety of computer programs known in the art. For example, computer programs such as BLAST2, BLASTN, BLASTP, Gapped BLAST, etc., can be used in some embodiments to generate alignments and/or to obtain a percent identity. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). In some embodiments, to obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. See the Web site having URL www.ncbi.nlm.nih.gov and/or McGinnis, S, and Madden, T L, W20-W25 Nucleic Acids Research, 2004, Vol. 32, Web server issue. Other suitable programs include CLUSTALW (Thompson J D, Higgins D G, Gibson T J, Nuc Ac Res, 22:4673-4680, 1994) and GAP (GCG Version 9.1; which implements the Needleman & Wunsch, 1970 algorithm (Needleman S B, Wunsch C D, J Mol Biol, 48:443-453, 1970.)

“Isolated” refers to a substance that is separated from at least some other substances with which it is normally found in nature, usually by a process involving the hand of man, or is artificially produced, e.g., chemically synthesized, or present in an artificial environment. In some embodiments, any of the nucleic acids, polypeptides, nucleic-acid-protein structures, protein complexes, or cells of the invention, is isolated. In some embodiments, an isolated nucleic acid is a nucleic acid that has been synthesized using recombinant nucleic acid techniques or in vitro transcription or chemical synthesis or PCR. In some embodiments, an isolated polypeptide is a polypeptide that has been synthesized using recombinant nucleic acid techniques or in vitro translation or chemical synthesis.

“Nucleic acid” is used interchangeably with “polynucleotide” and encompasses naturally occurring polymers of nucleosides, such as DNA and RNA, usually linked by phosphodiester bonds, and non-naturally occurring polymers of nucleosides or nucleoside analogs. In some embodiments a nucleic acid comprises standard nucleotides (abbreviated A, G, C, T, U). In other embodiments a nucleic acid comprises one or more non-standard nucleotides. In some embodiments, one or more nucleotides are non-naturally occurring nucleotides or nucleotide analogs. A nucleic acid can be single-stranded or double-stranded in various embodiments of the invention. A nucleic acid can comprise chemically or biologically modified bases (for example, methylated bases), modified sugars (2′-fluororibose, arabinose, or hexose), modified phosphate groups (for example, phosphorothioates or 5′-N-phosphoramidite linkages), locked nucleic acids, or morpholinos. In some embodiments, a nucleic acid comprises nucleosides that are linked by phosphodiester bonds. In some embodiments, at least some nucleosides are linked by a non-phosphodiester bond. A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications), non-standard nucleotides, delivery vehicles and approaches, etc., known in the art as being useful in the context of RNA interference (RNAi), aptamer, or antisense-based molecules for research or therapeutic purposes are contemplated for use in various embodiments of the instant invention. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. A nucleic acid may comprise a detectable label, e.g., a fluorescent dye, radioactive atom, etc. “Oligonucleotide” refers to a relatively short nucleic acid, e.g., typically between about 4 and about 60 nucleotides long. The terms “polynucleotide sequence” or “nucleic acid sequence” as used herein can refer to the nucleic acid material itself and is not restricted to the sequence information (i.e. the succession of letters chosen among the five base letters A, G, C, T, or U) that biochemically characterizes a specific nucleic acid, e.g., a DNA or RNA molecule. A naturally occurring nucleic acid or a nucleic acid identical in sequence to a naturally occurring nucleic acid may be referred to herein as a “native nucleic acid”, a “native XXX nucleic” (where XXX represents the name of the nucleic acid), or simply by the name of the nucleic acid or gene.

A “polypeptide” refers to a polymer of amino acids linked by peptide bonds. A protein is a molecule comprising one or more polypeptides. A peptide is a relatively short polypeptide, typically between about 2 and 60 amino acids in length. The terms “protein”, “polypeptide”, and “peptide” may be used interchangeably. A “multisubunit protein” is composed of multiple polypeptide chains physically associated with one another to form a complex. Polypeptides of interest herein often contain standard amino acids (the 20 L-amino acids that are most commonly found in nature in proteins). However, other amino acids and/or amino acid analogs known in the art can be used in certain embodiments of the invention. One or more of the amino acids in a polypeptide (e.g., at the N- or C-terminus or in a side chain) may be altered, for example, by addition, e.g., covalent linkage, of a moiety such as an alkyl group, carbohydrate group, a phosphate group, a halogen, a linker for conjugation, etc. A polypeptide sequence presented herein is presented in an N-terminal to C-terminal direction unless otherwise indicated. “Polypeptide domain” refers to a segment of amino acids within a longer polypeptide. A polypeptide domain may exhibit one or more discrete binding or functional properties, e.g., a binding activity or a catalytic activity. A domain may be recognizable by its conservation among polypeptides found in multiple different species. The term “polypeptide sequence” or “amino acid sequence” as used herein can refer to the polypeptide material itself and is not restricted to the sequence information (i.e. the succession of letters or three letter codes chosen among the letters and codes used as abbreviations for amino acid names) that biochemically characterizes a polypeptide. A naturally occurring polypeptide or a polypeptide identical in sequence to a naturally occurring polypeptide may be referred to herein as a “native polypeptide”, a “native XXX polypeptide” (where XXX represents the name of the polypeptide), or simply by the name of the polypeptide.

A “variant” of a nucleic acid refers to a nucleic acid that differs by one or more nucleotide substitutions, additions, or deletions, relative to a native nucleic acid. An addition can be an insertion within the nucleic acid or an addition at the 5′- or 3′-terminus. A deletion can be a deletion of a 5′-terminal region, 3′-terminal region and/or an internal region. A “variant” of a polypeptide refers to a polypeptide that differs by one or more nucleotide amino acid substitutions, additions, or deletions, relative to a native polypeptide. An addition can be an insertion within the polypeptide or an addition at the N- or C-terminus. A deletion can be a deletion of an N-terminal region, a C-terminal region, and/or an internal region. In some embodiments, the number of nucleotides or amino acids substituted in and/or added to a native nucleic acid or polypeptide or portion thereof can be for example, about 1 to 30, e.g., about 1 to 20, e.g., about 1 to 10, e.g., about 1 to 5, e.g., 1, 2, 3, 4, or 5. In some embodiments, the number of nucleotides or amino acids substituted in and/or added to a native nucleic acid or polypeptide or portion thereof can be for example, between 0.1% and 10% of the total number of nucleotides or amino acids in such native nucleic acid or polypeptide or portion thereof. In some embodiments, a variant comprises or consists of a nucleic acid or polypeptide whose sequence is at least 20%, 30%, 40%, 50%, 60%, 70%, 80%, 85%, 90%, 95%, 96%, 97%, 98%, 99%, 99.5%, or more identical in sequence to a native nucleic acid polypeptide (e.g., from a vertebrate such as a human, mouse, rat, cow, or chicken) over at least 50, 100, 150, 200, 250, 300, 400, 450, or 500 amino acids (but is not identical in sequence to native nucleic acid or polypeptide). In some embodiments, a variant comprises or consists of a nucleic acid or polypeptide at least 20%, 30%, 40%, 50%, 60%, 70%, 80%, 85%, 90%, 95%, 96%, 97%, 98%, 99%, 99.5%, or more identical in sequence to a native nucleic acid or polypeptide (e.g., from a vertebrate such as a human, mouse, rat, cow, or chicken) over at least 20%, 30%, 40%, 50%, 60%, 70%, 80%, 85%, 90%, 95%, 96%, 97%, 98%, 99%, 99.5%, or 100% of the native nucleic acid or polypeptide. In some embodiments, a variant nucleic acid or polypeptide comprises or consists of a fragment. A fragment is a nucleic acid or polypeptide that is shorter than a particular nucleic acid polypeptide and is identical in sequence to the nucleic acid polypeptide over the length of the shorter nucleic acid or polypeptide. In some embodiments, a fragment is at least 50%, 60%, 70%, 80%, 85%, 90%, 95%, 96%, 97%, 98%, or 99% as long as a native nucleic acid or polypeptide.

In some embodiments, a polypeptide fragment is an N-terminal fragment (i.e., it lacks a C-terminal portion of the native polypeptide). In some embodiments, a fragment is a C-terminal fragment (i.e., it lacks an N-terminal portion of the native polypeptide). In some embodiments, a fragment is an internal fragment, i.e., it lacks an N-terminal portion and a C-terminal portion of the native polypeptide. In some embodiments, a variant comprises two fragments fused together, e.g., an N-terminal portion and a C-terminal portion.

In some embodiments, a variant polypeptide comprises a heterologous polypeptide portion. The heterologous portion often has a sequence that is not present in the native polypeptide. In some embodiments, a heterologous portion has a sequence that is present in the native polypeptide, but at a different position. For example, a domain can be duplicated or positioned at a different location within the polypeptide. A heterologous polypeptide portion may be, e.g., between 5 and about 5,000 amino acids long, or longer, respectively, in various embodiments. Often it is between 5 and about 1,000 amino acids long. In some embodiments, a heterologous portion comprises a sequence that is found in a different polypeptide, e.g., a functional domain. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, 6×His, Flag, GST), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a variant has a tag located at the N- or C-terminus, e.g., as an N- or C-terminal fusion. The polypeptide could comprise multiple tags. In some embodiments, a 6×His tag and a NUS tag are present, e.g., at the N-terminus. In some embodiments, a tag is cleavable, so that it can be removed from the polypeptide, e.g., by a protease. In some embodiments, this is achieved by including a sequence encoding a protease cleavage site between the sequence encoding the portion homologous to a kinetochore polypeptide and the tag. Exemplary proteases include, e.g., thrombin, TEV protease, Factor Xa, PreScission protease, etc. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments, a heterologous portion comprises a detectable marker such as a fluorescent or luminescent protein, e.g., green, blue, sapphire, yellow, red, orange, and cyan fluorescent protein or derivatives thereof (e.g., EGFP, ECFP, EYFP), or monomeric red fluorescent protein or derivatives such as those known as “mFruits”, e.g., mCherry, mStrawberry, mTomato, or Cerulean or DsRed. In some embodiments, a heterologous portion comprises an enzyme that catalyzes a reaction leading to a detectable reaction product in the presence of a suitable substrate. Examples include alkaline phosphatase, beta galactosidase, horseradish peroxidase, luciferase, to name a few. Often, a detectable marker or reaction product is optically detectable, emitting or absorbing electromagnetic radiation (e.g., within the visible or nar infrared region of the spectrum) that can be observed visually and/or using suitable detection equipment. Detectable markers can include moieties that quench signals emitted from other moieties. In some embodiments, a heterologous portion comprises a selectable marker, e.g., a drug resistance marker or nutritional marker. Exemplary drug resistance markers include enzymes that inactivate compounds that would otherwise be cytotoxic or inhibit cell proliferation (e.g., neomycin or G418 resistance gene, puromycin resistance gene, blastocidin resistance gene etc.). A nutritional marker is typically an enzyme that permits a cell to survive in medium that lacks a particular nutrient. In some embodiments a tag or other heterologous portion is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, a domain that targets the polypeptide to a particular organelle, etc.

In some embodiments, a variant is a functional variant, i.e., the variant at least in part retains at least one biological activity of a native polypeptide, such as ability to bind to a particular molecule or structure, or ability to catalyze a biochemical reaction (or is a nucleic acid that encodes a functional variant polypeptide). One of skill in the art can readily generate functional variants or fragments. In some embodiments, a variant comprises one or more conservative amino acid substitutions relative to a native polypeptide. Conservative substitutions may be made on the basis of similarity in side chain size, polarity, charge, solubility, hydrophobicity, hydrophilicity and/or the amphipathic nature of the residues involved. As known in the art, such substitutions are, in general, more likely to result in a variant that retains activity as compared with non-conservative substitutions. In one embodiment, amino acids are classified as follows:

### Special: C

Neutral and small: A, G, P, S, T
Polar and relatively small: N, D, Q, E
Polar and relatively large: R, H, K
Nonpolar and relatively small: I, L, M, V
Nonpolar and relatively large: F, W, Y

### Special: C

See, e.g., Zhang, J. J. Mol. Evol. 50:56-68, 2000). In some embodiments, proline (P) is considered to be in its own group as a second special amino acid. Within a particular group, certain substitutions may be of particular interest, e.g., replacements of leucine by isoleucine (or vice versa), serine by threonine (or vice versa), or alanine by glycine (or vice versa). Of course non-conservative substitutions are often compatible with retaining function as well. In some embodiments, a substitution, deletion, or addition does not alter or delete or disrupt an amino acid or region of a polypeptide known or thought to be involved in or required for a particular activity that is desired to be maintained, while in other embodiments a substitution, deletion, or addition is selected to remove or disrupt a region known or thought be to in involved in or required for a particular activity. In some embodiments, an alteration is at an amino acid that differs among homologous polypeptides of different species. Variants could be tested in cell-free and/or cell-based assays to assess their activity.

In some embodiments, a variant or fragment that has substantially reduced activity as compared with the activity of native polypeptide (e.g., less than 10% of the activity of native polypeptide) is useful. For example, such polypeptide could interfere with the function of native polypeptide, e.g., by competing with native polypeptide, or serve as an immunogen for purposes of raising antibodies.

In some embodiments, a variant nucleic acid comprises a heterologous nucleic acid portion, which may be located at the 5′-terminus, 3′-terminus, or internally. The heterologous portion often has a sequence that is not present in the native nucleic acid. In some embodiments, a heterologous portion has a sequence that is present in the native nucleic acid, but at a different position. A heterologous nucleic acid portion may encode a heterologous polypeptide portion, such as any of those described above, or may not encode a polypeptide. A heterologous nucleic acid portion may or may not have a property or activity such as serving as an expression control element, recognition sequence for a DNA binding protein, or encoding a functional RNA.

As used herein, the term “purified” refers to agents or entities (e.g., compounds such as polypeptides, nucleic acids, small molecules, etc.) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure. In some embodiments, a nucleic acid or polypeptide is purified such that it constitutes at least 75%, 80%, 855%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed. In some embodiments, any of the nucleic acids, polypeptides, nucleic-acid-protein structures, or protein complexes of the invention, is at least partly purified.

A “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

A “subject” can be any multicellular animal, e.g., a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. In some embodiments, the animal is a mammal of economic importance, such as a cow, horse, pig, goat, or sheep.

“Treat”, “treating” and similar terms refer to providing medical and/or surgical management of a subject. Treatment can include, but is not limited to, administering a compound or composition (e.g., a pharmaceutical composition or a composition comprising appropriate cells in the case of cell-based therapy) to a subject. Treatment is typically undertaken in an effort to alter the course of a disorder (which term is used to refer to a disease, syndrome, or abnormal condition) or undesirable or harmful condition in a manner beneficial to the subject. The effect of treatment can generally include reversing, alleviating, reducing severity of, delaying the onset of, curing, inhibiting the progression of, and/or reducing the likelihood of occurrence or reoccurence of the disorder or condition, or one or more symptoms or manifestations of such disorder or condition. A composition can be administered to a subject who has developed a disorder or is at risk of developing a disorder. A composition can be administered prophylactically, i.e., before development of any symptom or manifestation of a disorder. Typically in this case the subject will be at increased risk of developing the disorder relative to a member of the general population. For example, a composition can be administered to a subject with a risk factor, e.g., a mutation in a gene, wherein the risk factor is associated with increased likelihood of developing the disorder but before the subject has developed symptoms or manifestations of the disorder. “Preventing” can refer to administering a composition to a subject who has not developed a disorder, so as to reduce the likelihood that the disorder will occur or so as to reduce the severity of the disorder should it occur. The subject may be identified (e.g., diagnosed by a medical practitioner) as having or being at risk of developing the disorder (e.g., at increased risk relative to many most other members of the population or as having a risk factor that increases likelihood of developing the disorder).

### II. Modified Kinetochore Components, Engineered Kinetochores, Artificial Chromosomes, Cells, and Transgenic Animals

The kinetochore is a proteinaceous structure that assembles on chromosomes in eukaryotic organisms and mediates their attachment to spindle microtubules. The region of chromosomal DNA at which kinetochore assembly occurs is termed the “centromere”. In vertebrate cells, the centromere typically contains large arrays of repetitive DNA. In humans, the major centromeric repeat unit is called α-satellite DNA, although a number of other repeat units are found in vertebrate centromeres. Three distinct regions of vertebrate kinetochores have been described: the inner kinetochore, which forms the interface with chromatin, the outer kinetochore, which provides the binding site for spindle microtubules, and the central kinetochore, which is the region between the inner and outer kinetochores (reviewed in Cheeseman & Desai, 2008).

A large number of kinetochore proteins (also termed “kinetochore components” herein” have been identified. CENP-A (where “CENP” stands for “centromere protein”) is a variant of histone H3, one of the core subunits of nucleosomes. The deposition of nucleosomes containing CENP-A (also called CenH3) occurs predominantly at centromeres, and is required for kinetochore assembly at these sites (Howman et al., 2000). In addition to CENP-A, a group of at least proteins (CENP-H, I, and K-W) termed the Constitutive Centromere Associated Network (CCAN), also called the CENP-ANAC/CAD, is constitutively present at centromeres in human cells throughout the cell cycle (Foltz et al., 2006; Okada et al., 2006; reviewed in Cheeseman and Desai, 2008; see also Amano et al., 2009 describing CENP-X as an additional CCAN component, i.e., these molecules are detectably present at or associated with the centromere in significant amounts throughout the cell cycle. Outer kinetochore proteins are assembled at kinetochores beginning at prophase and leave kinetochores at the end of mitosis. The KNL1/Mis12 complex/Ndc80 complex (KMN) proteins (also termed the KMN network) assemble stably within the outer kinetochore to produce core attachment sites for MTs. The Mis12 complex comprises Mis12, Dsn1, Nnf1, and Nsl1 proteins, while the Ndc80 complex comprises Ndc80, Nuf2, Spc24, and Spc25 proteins. Other outer kinetochore components transiently associate with kinetochores after entry into mitosis and include spindle assembly checkpoint (SAC) components, MT motor proteins, the large coiled-coil protein CENP-F, and MT-binding proteins that regulate plus-end assembly dynamics (Musacchio and Salmon, 2007; Santaguida and Musacchio; 2009).

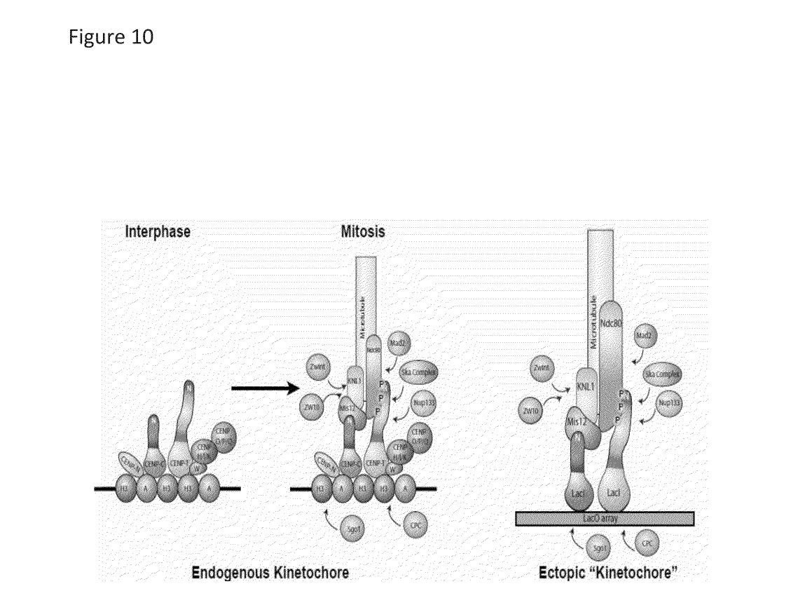

The invention relates in part to the discovery that that the kinetochore proteins CENP-C and CENP-T are sufficient for assembly of a minimal kinetochore structure in human cells. As described in further detail in the Examples, modified human CENP-C and CENP-T polypeptides, in which a C-terminal portion containing the endogenous DNA-binding domain(s) was removed and the remaining (N-terminal) portion of the polypeptide was fused to the LacI protein, were generated. (As known in the art, LacI is a bacterial protein that binds to a DNA segment termed the Lac operator (LacO) in a sequence-specific manner.) The modified human CENP-C and CENP-T polypeptides also contained mCherry or green fluorescent protein (GFP) as an N-terminal domain, which allowed convenient visualization of these polypeptides using fluorescence microscopy. Modified CENP-C and CENP-T polypeptides were expressed in human cells that contained multiple LacO sequences integrated into one chromosome. When examined using fluorescence microscopy, the modified CENP-C and CENP-T polypeptides were visualized as a single focus in transected cells, consistent with their binding to the LacO sequences. KNL1, the Mis12 complex subunit Dsn1, and the Ndc80 complex subunit Ndc80 co-localized with the modified CENP-C and CENP-T polypeptides, as did a number of other kinetochore proteins including Zwint, Nup133, Ska1, and components of the Chromosome Passenger Complex (CPC) such as Aurora B kinase. The structure assembled substantially in the absence of the centromere specific histone H3 variant CENP-A, as determined by immunostaining with antibodies to CENP-A, and exhibited kinetochore function based at least on the presence of representative kinetochore components, interaction with microtubules, and the segregation behavior of chromosomes containing the foci. FIG. 10 presents a diagram showing an exemplary engineered kinetochore according to certain embodiments of the invention and illustrates interactions of various kinetochore components according to certain embodiments of the invention.

In some aspects, the invention provides modified kinetochore polypeptides. As used herein a “modified polypeptide” differs from a native polypeptide in terms of amino acid sequence or has a non-polypeptide portion attached thereto, wherein the non-polypeptide portion is not attached to the native polypeptide as found in nature. In some embodiments, a modified polypeptide is a variant of a native kinetochore polypeptide. In some embodiments of the invention, the modified kinetochore polypeptide is a modified CCAN polypeptide. Certain CCAN polypeptides, such as CENP-C and CENP-T, are capable of binding to DNA. A CCAN polypeptide capable of binding to DNA is referred to as a “DNA-binding CCAN” (DBCCAN)). In some embodiments, the modified CCAN polypeptide is a modified DBCCAN polypeptide. In some embodiments, the modified DBCCAN polypeptide is a modified CENP-C polypeptide. In other embodiments, the modified DBCCAN polypeptide is a modified CENP-T polypeptide. In yet other embodiments, the modified DBCCAN polypeptide is a modified CENP-W polypeptide.

One of skill in the art will also readily be able to obtain amino acid sequences of kinetochore polypeptides, and the genomic and mRNA sequences encoding them, from publicly available databases, such as those available at the National Center for Biotechnology Information (NCBI; www.ncbi.nlm.nih.gov), e.g., Gene, GenBank, Proteins, etc. For example, the Gene database provides sequence information (e.g., accession numbers for reference sequences (in the RefSeq database)) and functional information, which can be obtained, e.g., by searching on a name or Gene ID for a gene or protein of interest. In addition, kinetochore protein sequences, and nucleic acid constructs encoding them, are described in the scientific literature and available from a variety of sources. One of skill in the art can readily generate modified kinetochore polypeptides as described herein. Table 1 provides a list of the official symbol and Gene ID of certain human kinetochore polypeptides of interest. While vertebrate kinetochore proteins have been most extensively studied in certain vertebrates of interest such as human, mouse, and chicken, homologs of many of the proteins found in these animals have been identified across a wide range of vertebrate species. The various aspects of the invention encompass embodiments applicable to any vertebrate kinetochore polypeptide of interest. It will be appreciated that multiple alleles of a gene may exist among individuals of the same species due to natural allelic variation. For example, differences in one or more nucleotides (e.g., up to about 1%, 2%, 3-5% of the nucleotides) of the nucleic acids encoding a particular protein may exist among individuals of a given species. Due to the degeneracy of the genetic code, such variations frequently do not alter the encoded amino acid sequence, although DNA polymorphisms that lead to changes in the amino acid sequences of the encoded proteins can exist. It will also be understood that multiple isoforms of certain proteins encoded by the same gene may exist as a result of alternative RNA splicing or editing. It should be understood that the invention provides embodiments relating to such allelic variants or isoforms as applicable. Examples of polymorphic variants can be found in, e.g., the Single Nucleotide Polymorphism Database (dbSNP) (available at the NCBI website at www.ncbi.nlm.nih.gov/projects/SNP/), which contains single nucleotide polymorphisms (SNPs) as well as other types of variations (see, e.g., Sherry S T, et al. (2001). “dbSNP: the NCBI database of genetic variation”. Nucleic Acids Res. 29 (1): 308-311; Kitts A, and Sherry S, (2009). The single nucleotide polymorphism database (dbSNP) of nucleotide sequence variation in The NCBI Handbook [Internet]. McEntyre J, Ostell J, editors. Bethesda (MD): National Center for Biotechnology Information (US); 2002 (www.ncbi.nlm.nih.gov/bookshelf/br.fcgi?book=handbook&part=ch5).

TABLE 1
Human genes encoding selected kinetochore polypeptides

| Name | Official Symbol | Gene ID |
|---|---|---|
| centromere protein A | CENP-A | 1058 |
| centromere protein C1 | CENP-C1 (also called CENP-C) | 1060 |
| centromere protein T | CENP-T | 80152 |
| centromere protein W | CENP-W | 387103 |
| cancer susceptibility | CASC5 (also called KNL1) | 57082 |
| candidate 5 | | |
| MIS12 | Mis12 | 79003 |
| NDC80 Homolog | NDC80 (also called HEC1) | 10403 |
| NUF2 | NUF2 | 83540 |
| SPC24 | SPC24 | 147841 |
| SPC25 | SPC25 | 57405 |
| ZW10 interactor | ZWINT | 11130 |
| Sgo1 | SGOL1 | 151648 |
| Aurora B kinase | AURKB | 9212 |
| ZW10 | ZW10 | 9183 |
| Ska3 | SKA3 (also called RAMA1) | 221150 |
| Mad2 | MAD2L1 | 4085 |
| Mad2 | MAD2L2 | 10459 |

In some embodiments, a modified kinetochore polypeptide of the invention comprises a heterologous DNA binding domain. As used herein, a “DNA binding domain” (DBD) is a polypeptide or portion thereof that binds to single or double stranded DNA. The term “heterologous” is used to indicate that the DBD is not present within the native form of the polypeptide. A polypeptide that contains a DBD can be tethered to DNA via physical interaction of the DBD with DNA. In some embodiments, a DBD comprises or consists of an independently folded protein domain. In some embodiments, a DBD of use in the invention can recognize one or more specific DNA sequences (sometimes termed a “recognition sequence”). Such a DBD binds preferentially to its recognition sequence as compared with its binding to other DNA sequences. For example, the affinity of such a DBD for a DNA segment containing a recognition sequence can be, e.g., at least 10-fold, 100-fold, 1.000-fold or more greater than its affinity for random DNA sequences. A DBD and a DNA segment that contains a recognition sequence for the DBD (or the recognition sequence itself) are termed “cognate” to one another. In some embodiments, the K_{d} for binding of a DBD to a cognate DNA segment is less than about 10⁻⁴ M, e.g., less than about 10⁻⁵ M, less than about 10⁻⁶ M, less than about 10⁻⁷ M, less than about 10⁻⁸ M, less than about 10⁻⁹, less than about 10⁻¹⁰ M, less than about 10⁻¹¹ M less than about 10⁻¹² M, or less than about 10⁻¹³ M. In some embodiments, a DBD can have a general affinity to DNA. Types of DBDs include, for example, helix-turn-helix, helix-loop-helix, zinc finger, leucine zipper, winged helix, winged helix turn helix, HMG-box, immunoglobulin fold, B3 domain, and TAL effector DBD. A large number of DBDs, and their cognate recognition sequences, are known in the art and can be used in various embodiments of the invention. Naturally occurring DBDs are found in prokaryotic and eukaryotic organisms, e.g., bacteria, fungi (e.g., yeast), plants, invertebrates (e.g., insects), and vertebrates (e.g., mammals, avians). In some embodiments of the invention, a naturally occurring DBD is used. Often, such DBDs occur in proteins involved in regulation of gene expression, e.g., transcriptional regulators. In some embodiments, a DBD of use in the present invention is found in a transcriptional activator. In some embodiments, a DBD of use in the present invention is found in a transcriptional repressor. In some embodiments, a DBD from a prokaryotic transcriptional regulator is used. In some embodiments, a full length naturally occurring DBD-containing protein is used. In other embodiments, a DBD-containing fragment or variant is used. For example, a transcriptional activation or repression domain may be deleted. Exemplary prokaryotic transcriptional regulator families include, e.g., the LysR, AraC/XylS, TetR, LuxR, LacI/GalR, ArsR, IcIR, MerR, AsnC, MarR, NtrC, OmpR, DeoR, cold shock, GntR, and Crp families. See, e.g., Swint-Kruse, L and Matthews, K (2009). Allostery in the LacI/GalR family: variations on a theme, Current Opinion in Microbiology, 12(2): 129-137 Wilson, C J, et al. (2007). The lactose repressor system: paradigms for regulation, allosteric behavior and protein folding. Cellular and Molecular Life Sciences, 64(1), 3-16, Culard, F., et al., (1987) Lac repressor-Lac operator complexes, Eur. Biophys. J. 14: 169-178, and Ramos, J L, et al., (2005) The TetR Family of Transcriptional Repressors, Microbiol. & Mol. Biol. Rev., 69(2): 326-356, and references in the foregoing articles, for further information. In some embodiments, a prokaryotic DBD is found in a bacterium, e.g., a gram positive or gram negative bacterium. In some embodiments, a DBD from E. coli is used. In some embodiments of particular interest, the lactose repressor protein (LacI), or a DBD-containing portion thereof, is used. LacI binds to its cognate recognition sequence (LacO) with a very high affinity (estimated K_{d} 10⁻¹³ M). In other embodiments of particular interest, a tet repressor protein (TetR) or a DBD-containing portion thereof, is use. In other embodiments, the DBD originates from a fungus, e.g., a yeast, e.g., S. cerevesiae. In some embodiments, a DBD from a fungal transcriptional regulator is used. For example, the DBD of the yeast Gal4 protein, which in the case of S. cerevesiae Gal4 binds to a sequence termed the UAS (consensus sequence CGG-N11-CCG, where N can be any base), could be used in certain embodiments. In some embodiments, a DBD from a vertebrate, e.g., a mammal, is used. For example, DBDs from a nuclear receptor, e.g., a steroid hormone receptor, may be used in certain embodiments.

One of skill in the art will readily be able to obtain sequences of numerous DBDs and cognate DNA recognition sequences. In addition to NCBI databases, a number of databases that focus on DNA binding proteins and/or their recognition sequences could be used. For example, DNA-binding Domain (DBD) is a database of predicted sequence-specific DNA-binding transcription factors (TFs) for all publicly available proteomes (Wilson, D., et al., (2008) DBD—taxonomically broad transcription factor predictions: new content and functionality. D88-D92 Nucleic Acids Research, Vol. 36, Database issue Published online 11 Dec. 2007). Access to the DBD database is via http://transcriptionfactor.org. The Transcription Factor Database (accessed at http://cmgm.stanford.edu/help/manual/databases/tfd.html) is a database of DNA recognition sequences for eukaryotic and prokaryotic sequence-specific transcription factors. The JASPAR CORE database (accessed at http://jaspar.genereg.net/) contains a curated, non-redundant set of profiles, derived from published collections of experimentally defined transcription factor binding sites for eukaryotes. It will be understood that a given DBD may tolerate some variability in the sequence of the DNA segment to which it binds. Consensus recognition sequences for a number of DBDs are known and can be used in various embodiments of the invention, or such consensus sequences can be identified. In some embodiments, a DBD binds to a consensus recognition sequence that is not naturally present within the genome of a vertebrate. A vertebrate can be, e.g., a human, non-human primate, rodent (e.g., mouse, rat, rabbit), ungulate, avian (e.g., chicken), etc., in various embodiments of the invention. It should be noted that wherever the term “vertebrate” is used herein as a noun or adjective as in “vertebrate cell”, “vertebrate gene”, etc., the invention encompases embodiments that pertain to any vertebrate, e.g., a human, non-human primate, rodent (e.g., mouse, rat, rabbit), ungulate, avian (e.g., chicken), etc., unless otherwise indicated or evident from the context. In some embodiments, a vertebrate species has a genome that has been sequenced and/or in which a CENP-C and/or CENP-T gene and/or polypeptide have been identified and, optionally, the coding sequence determined. In some embodiments of the invention, a variant of a naturally occurring DBD and/or a variant of a naturally occurring DBD recognition sequence is used. In some embodiments, an artificially designed DBD and/or a variant of a naturally occurring recognition sequence is used.

In some embodiments of the invention, a modified kinetochore polypeptide, e.g., a modified DBCCAN, comprises a non-polypeptide DNA binding moiety which, in some embodiments, binds to DNA in a sequence-specific manner. For example, an oligonucleotide (e.g., an aptamer) or a small molecule having sequence-specific DNA binding properties can be used in certain embodiments of the invention. In some embodiments, a peptide nucleic acid, triplex nucleic acid, or polyamide is used. In some embodiments, a DNA binding moiety is conjugated to a polypeptide using any of a variety of conjugation methods known in the art. See, e.g., Hermanson, G., Bioconjugate Techniques, 2^{nd} ed., Academic Press, 2008.

In some embodiments of particular interest, a modified kinetochore polypeptide of the invention is a modified DBCCAN polypeptide, wherein the modified DBCCAN polypeptide comprises a heterologous DBD such as any of those described above. In some embodiments, a modified DBCCAN polypeptide that comprises a heterologous DNA binding domain lacks at least a portion of the region of the native DBCCAN that is required for binding of the DBCCAN to DNA (e.g., total genomic DNA or alpha satellite DNA of proliferating cells of the type in which the DBCCAN occurs naturally). For example, in some embodiments, a modified human CENP-C polypeptide lacks at least a central domain and/or at least a C-terminal domain of CENP-C that mediates binding to DNA (see, e.g., Song K, et al. (2002). Mutational analysis of the central centromere targeting domain of human centromere protein C, (CENP-C), Exp Cell Res. 275(1):81-91; Trazzi S, et al., (2009) The C-terminal domain of CENP-C displays multiple and critical functions for mammalian centromere formation. PLoS One. 4(6): e5832. In some embodiments, a modified human CENP-T polypeptide lacks at least a C-terminal portion of CENP-T, e.g., at least a portion of amino acids 474-639 of CENP-T (see, e.g., Hori, 2008). In some embodiments, at least part of a histone fold domain found in the native polypeptide is absent. In some embodiments, a region of a DBCCAN required for DNA binding is altered, so that it is no longer capable of binding to DNA. In some embodiments, binding of a modified DBCCAN of the invention to DNA would (in the absence of the heterologous DBD) be substantially reduced relative to binding of the native DBCCAN to such DNA. In some embodiments, binding to DNA may be reduced by at least 5, 10, 20, 50-fold or more, or to levels not detectably greater than background levels.

In some embodiments, binding of a DBD to a cognate DNA segment is regulatable, e.g., by a small molecule or protein. For example, the presence or absence of a small molecule or protein may inhibit, induce, or enhance binding of a DBD to a cognate DNA segment, regulate localization of a polypeptide comprising a DBD, and/or regulate transcription of mRNA encoding a polypeptide comprising a DBD. In some embodiments, the small molecule is tetracycline or a tetracycline derivative or analog (e.g., doxycycline), and the DBD is a TetR DBD. In some embodiments, the small molecule is lactose or a derivative or analog thereof, and the DBD is a LacI DBD. For example, a lactose derivative can be a substituted galactoside, where the glucose moiety of lactose is replaced by another chemical group. Isopropyl-β-D-thio-galactoside (IPTG) is frequently used as an inducer of the lac operon. IPTG binds to Lac repressor and inactivates it. Thus, binding of a polypeptide comprising LacR to the lac operator would be inhibited. For uses in cells, a compound that is reasonably cell-permeable may be selected. In yet other embodiments, a small molecule is a steroid hormone or derivative or analog thereof. A derivative or analog may be naturally occurring or artificial. In some embodiments, a non-kinetochore polypeptide (whose expression or activity is optionally regulated by a small molecule) binds to the cognate DNA segment, thereby competing with binding of the modified DBCCAN and thus inhibiting assembly of an engineered kinetochore.

The invention contemplates use of various polypeptides of the Tet-Off, Tet-On, Tet-On Advanced transactivator (also known as rtTA2S-M2), and/or Tet-On 3G (also known as rtTA-V 10) systems or portions thereof lacking a transactivation domain as heterologous portions of a modified DBCCAN. (See, e.g., See, e.g., Bujard, Hermann & Gossen, M. (1992). Tight Control of Gene Expression in Mammalian Cells by Tetracycline-Responsive Promoters. Proc. Natl. Acad. Sci. U.S.A. 89 (12): 5547-51; Allen, N, et al. (2000). “Directed Mutagenesis in Embryonic Stem Cells”. Mouse Genetics and Transgenics: 259-263; Urlinger, S, et al. (2000). Exploring the sequence space for tetracycline-dependent transcriptional activators: Novel mutations yield expanded range and sensitivity. Proc. Natl. Acad. Sci. U.S.A. 97 (14): 7963-8; Zhou, X, et al (2006). Optimization of the Tet-On system for regulated gene expression through viral evolution. Gene Ther. 13 (19): 1382-1390.) The use of a regulatable DBD can allow the binding of a modified DBCCAN to DNA and/or the assembly of additional kinetochore components therwith, to be inhibited, induced, or enhanced. Thus assembly or use of an engineered kinetochore or artificial chromosome comprising a site for assembly of an engineered kinetochore can be conditional in some embodiments of the invention.

In some embodiments, a modified DDCCAN kinetochore polypeptide of the invention comprises a sequence sufficient to bind to at least one KMN network polypeptide and to recruit such KMN polypeptide to DNA containing a cognate DNA segment. In some embodiments, a modified DDCCAN kinetochore polypeptide of the invention comprises a sequence sufficient to bind to at least one non-DNA binding CCAN and to recruit such non DNA-binding CCAN to DNA containing a cognate DNA segment. As described in the Examples, a modified CENP-C polypeptide comprising amino acids 1-235 of human CENP-C fused to LacI and a modified CENP-T polypeptide comprising amino acids 1-242 of human CENP-T fused to LacI were shown to bind to DNA containing multiple copies of LacO and did not exhibit detectable binding at other locations in genomic DNA. It would be expected that smaller deletions would also result in substantial loss of DNA binding activity, and the use of modified human CENP-C and human CENP-T polypeptides having such smaller deletions is within the scope of the invention. It would also be expected that smaller fragments of human CENP-C, e.g., fragments lacking at least some N-terminal and/or C-terminal amino acids of the 1-235 fragment of human CENP-C, would retain ability to participate in recruiting additional kinetochore components, and the use of modified human CENP-C polypeptides lacking at least some N-terminal and/or C-terminal amino acids of the 1-235 fragment of human CENP-C is within the scope of the invention. Similarly, it would be expected that smaller fragments of human CENP-T, e.g., fragments lacking at least some N-terminal and/or C-terminal amino acids of the 1-242 fragment of human CENP-T, would retain ability to participate in recruiting additional kinetochore components, and the use of modified CENP-T polypeptides lacking at least some N-terminal and/or C-terminal amino acids of the 1-242 fragment of human CENP-T is within the scope of the invention. In some embodiments, a modified CENP-C polypeptide or modified CENP-T polypeptide comprises amino acids 10-150, 25-150, 50-150, 75-150, 10-175, 25-175, 50-175, 75-175, 10-200, 25-200, 50-200, 75-200, 10-225, 25-225, 50-225, or 75-225 of human CENP-C or human CENP-T. In some embodiments, a modified human CENP-C polypeptide or modified human CENP-T polypeptide comprises a variant of amino acids 10-150, 25-150, 50-150, 75-150, 10-175, 25-175, 50-175, 75-175, 10-200, 25-200, 50-200, 75-200, 10-225, 25-225, 50-225, or 75-225 of human CENP-C or human CENP-T. In some embodiments, a variant may comprise a polypeptide least 95%, 96%, 97%, 98%, 99% or 100% identical to amino acids 10-150, 25-150, 50-150, 75-150, 10-175, 25-175, 50-175, 75-175, 10-200, 25-200, 50-200, 75-200, 10-225, 25-225, 50-225, or 75-225 of human CENP-C or human CENP-T. Shorter and longer fragments of human CENP-C and human CENP-T, and variants thereof, are within the scope of the invention. In some embodiments the invention provides a modified non-human vertebrate CENP-C or CENP-T polypeptide, which is homologous to any of the afore-mentioned fragments of human CENP-C or CENP-T polypeptide (or is a variant of a homologous fragment). In some embodiments, the modified non-human vertebrate CENP-C or CENP-T polypeptide does not significantly bind to DNA in the absence of a heterologous DBD and has the ability to bind to and recruit a non DNA-binding kinetochore polypeptide, such as a KMN component, to DNA. Homologous fragments of non-human vertebrate DBCCAN polypeptides can be identified, e.g., based on sequence alignment or functional assays. In some embodiments, a modified non-vertebrate DBCANN polypeptide does not necessarily have sequence homology to a human DBCANN polypeptide. However, it can have properties such as inability to bind significantly to DNA in the absence of a heterologous DBD and ability to bind to and recruit a non DNA-binding kinetochore polypeptide to DNA.

In some aspects, the invention provides modified CENP-C and modified CENP-T polypeptides, wherein the modified CENP-C and modified CENP-T polypeptides comprise different DBDs. For purposes of description, LacI and TetR will be used as exemplary DBDs. For example, a modified CENP-C polypeptide may comprise LacI, and a modified CENP-T polypeptide may comprise TetR, or vice versa. In such embodiments, a suitable DNA segment for binding both modified CENP-C and modified CENP-T polypeptides comprises a cognate DNA segment for LacI and a cognate DNA segment for TetR, e.g., LacO and TetO sequences. In some embodiments, a cognate DNA segment comprises a response element of a gene whose expression is at least in part regulated by LacI or TetR (or a different polypeptide comprising a DBD). For example, a tetracycline response element (TRE) consists of 7 repeats of the 19 bp bacterial TetO sequence separated by spacer sequences. Such a TRE may be used in certain embodiments of the invention. The cognate DNA segments for the different DBDs may be located adjacent to one another, may be alternated, grouped, or be interspersed among other DNA segments that do not contain recognition sequences for the respective DBDs. For example, in some embodiments, LacO and TetO alternate with one another, while in other embodiments, there may be multiple LacO segments followed by multiple TetO segments, and such arrangements may be repeated multiple times. For example, a DNA segment may be represented generally as: [(LacO)ₘ(Xaa)ₖ(TetO)ₙ], where k m, k, and n are non-negative integers; either m or n is at least 1; Xaa represents independently any amino acid, and the [(LacO)ₘ(Xaa)ₖ(TetO)ₙ] unit can be repeated multiple times with the same or different values of m, k and n in the various units. In some embodiments, m=0 throughout the DNA segment, in which case modified kinetochore polypeptides to be targeted to the DNA segment should comprise a heterologous DBD that binds to TetO, while in other embodiments n=0 throughout the DNA segment, in which case modified kinetochore polypeptides to be targeted to the DNA segment should comprise a heterologous DBD that binds to LacO. In some embodiments, m and n are equal, while in other embodiments, they are different. They may be independently selected, or a defined ratio of m:n may be used. For example, in some embodiments, it may be desirable to select m and n so that approximately the same number of modified CENP-C and modified CENP-T polypeptides bind to a DNA segment. In some embodiments, it may be desirable to select m and n so that more modified CENP-C polypeptides than modified CENP-T polypeptides bind to the DNA segment, or vice versa. In some embodiments, m and n are each between 1 and 300, e.g., between 10 and 200, e.g., between 50 and 100 or between 100 and 200 or between 200 and 300. In some embodiments, k is 0, while in some exemplary embodiments k is between 1-100. The selection of optimum values of m, k, and n, and/or the identity of the amino acids represented as Xaa, for a particular purpose can be achieved, e.g., by systematically varying these values and identities and assessing a parameter of interest, such as recruitment of one or more additional kinetochore components, microtubule binding, deformation of a DNA segment by microtubules, segregation behavior of DNA (e.g., a chromosome) containing the DNA segment, etc. In some embodiments, amino acids are selected for (Xaa)ₖ that do not form a predicted secondary structure or motif such as a helix or turn and that do not constitute a known functional domain that might interact with other DNA or with cellular proteins. Suitable DNA segments can be prepared, e.g., using conventional cloning and/or amplification methods.

The invention provides nucleic acids encoding one or more modified DBCCAN polypeptides of the invention. In some embodiments, to the extent a modified DBCCAN polypeptide is identical to a native DBCCAN polypeptide, the naturally occurring nucleic acid sequence encoding the native DBCCAN polypeptide may be used. The sequence encoding a native DBCCAN can be altered appropriately to encode the modified DBCCAN. However, one of skill in the art will appreciate that due to the degeneracy of the genetic code, a large number of different nucleic acid sequences can encode any desired polypeptide, and such sequences can be used in the invention. In some embodiments, a sequence is codon optimized for expression in a host cell or organism of interest. In some embodiments, a nucleic acid comprises a portion that encodes a modified DBCCAN polypeptide, operably linked to expression control element(s) such as a promoter, promoter/enhancer, etc., so that the modified DBCCAN is produced in cells under appropriate conditions. In some embodiments, a nucleic acid of the invention is contained in a vector, e.g., a plasmid, virus vector, etc. In some embodiments, the invention provides a nucleic acid containing a first region that encodes a first modified DBCCAN and a second region that encodes a second modified DBCCAN. For example, the invention provides a nucleic acid containing a first region that encodes a modified CENP-C polypeptide and a second region that encodes a modified CENP-T polypeptide. In some embodiments, the nucleic acid further comprises a DNA segment to which the modified CENP-C and modified CENP-T polypeptides bind, e.g., via heterologous DBDs. For example, if the modified DBCCAN polypeptides comprise LacO and/or TetR DBDs, the nucleic acid may comprise one or more [(LacO)ₘ(Xaa)ₖ(TetO)ₙ] units.

In some embodiments, a nucleic acid of the invention encodes one or more modified DBCCAN polypeptides of the invention and, optionally, comprises a DNA segment cognate to the encoded DBCCAN polypeptide(s). In some embodiments, the nucleic acid further comprises a portion of interest. The term “portion of interest” is used to refer to a portion of a nucleic acid that can have any sequence in various embodiments of the invention. The nucleic acid portion can be selected by an individual practicing the invention. The sequence of the portion may or may not be completely or partially known to the individual. In some embodiments, it is randomly selected. In some embodiments, a portion of interest could comprise or consist of a random fragment of genomic DNA, e.g., chemically, physically, or enzymatically fragmented or cleaved DNA. In some embodiments, the portion of interest comprises at least 10 kB of vertebrate genomic DNA, e.g., between 10 kB and about 250 Mb in various embodiments. For example, the nucleic acid may comprise a portion of interest between about 100 kB and about 50 Mb, e.g., between 1 Mb and 5 Mb, or between 5 Mb and 10 Mb, or between 10 Mb and 20 Mb, or between 20 Mb and 50 Mb, or between 50 Mb and 100 Mb of vertebrate genomic DNA in various embodiments. In some embodiments, the amount of genomic DNA is approximately the same amount as is present in a naturally occurring chromosome of a vertebrate of interest, e.g., human, rodent, or avian. In some embodiments, the vertebrate genomic DNA comprises a gene, which includes a sequence that is transcribed (which can include both exons and introns in the case of a gene that encodes a polypeptide) and, in some embodiments, one or more endogenous expression control element(s). Without wishing to be bound by any theory, expressing a gene using endogenous expression control elements may permit expression that more closely replicates a naturally occurring cell or tissue-specific expression pattern or alternative splicing pattern and/or appropriate transcriptional response to physiologic, pathologic, or developmental signals or inputs than expression using heterologous expression control elements. In some embodiments, the nucleic acid portion, e.g., vertebrate genomic DNA, comprises a sequence that is transcribed to produce a non-coding RNA (e.g., a functional RNA (or precursor thereof)). In some embodiment, a functional RNA regulates a cellular process or property such as transcription, translation, mRNA degradation, etc. In some embodiments, a functional RNA is a microRNA, short interfering RNA, short hairpin RNA, long non-coding RNA, transfer RNA, ribosomal RNA, antisense RNA, or ribozyme.

In some embodiments, the nucleic acid portion, e.g., vertebrate genomic DNA, comprises a sequence that is transcribed to yield an RNA that encodes a polypeptide. In some embodiments, the gene is one whose mutation, deletion, or altered expression is at least in part responsible for causing a disorder (e.g., in humans). In some embodiments, mutation, alteration, or altered expression (which may be due to a mutation or deletion) of a single gene can cause the disorder. Exemplary genetic disorders and the associated proteins include, e.g., metabolic enzyme deficiencies such as Krabbe's disease (galactocerebrosidase), immunodeficiencies such as severe combined immunodefiency (adenosine deaminase gene), hemophilia (Factor VII, Factor VIII), various forms of muscular dystrophy, e.g., Duchennne and Becker muscular dystrophy (dystrophin), cystic fibrosis (cystic fibrosis transmembrane conductance regulator), Freidrich's ataxia (frataxin). In some embodiments, a gene encodes a structural protein such as collagen or another extracellular matrix protein. One of skill in the art will be aware of numerous additional genetic diseases and associated genes. In some embodiments, genomic DNA comprises an immunoglobulin locus, e.g., a heavy chain or light chain (e.g., kappa or lambda) locus, or a T cell receptor gene. In some embodiments a gene comprises exons that are located over at least 20 kB of genomic DNA, e.g., over 20-100 kB of genomic DNA or more. In some embodiments, the genomic DNA comprises multiple genes, e.g., 2, 3, 4, 5, or more genes. In some embodiments, genomic DNA comprises multiple microRNA genes. In some embodiments, genomic DNA comprises multiple genes that encode polypeptides that form a multiprotein complex or function in the same biological pathway. For example, many enzymes and channels are composed of multiple different subunits.

In some embodiments, a nucleic acid of the invention comprises a telomeric sequence. As known in the art, telomeres are regions of repetitive DNA at the end of a linear chromosome, which protect the end of the chromosome from shortening. They typically comprise arrays of guanine-rich, six- to eight-base-pair-long repeats, often terminating with a 3′ single-stranded-DNA overhang. An exemplary telomere repeat element is TTAGGG. In some embodiments, the telomeric sequence comprises (TTAGGG)ₙ, wherein n is at least 3, e.g., between 3 and 1000. In some embodiments, a telomere sequence is between about 3 and 20 kB long. In some embodiments, a nucleic acid of the invention comprises a vertebrate (e.g., human, rodent, chicken) origin of replication. Vertebrate origins of replication are located throughout the genome and can be obtained from a sufficiently long segment of genomic DNA. In some embodiments, a nucleic acid of the invention comprises a sequence that recruits one or more polypeptides that function in chromosome condensation and/or one or more polypeptide components of cohesin, the protein complex responsible for binding the sister chromatids during S phase, through G2 phase, and into M phase. For example, the nucleic acid can contain a CCCTC-binding factor (CTCF) recognition sequence. In some embodiments, the nucleic acid can contain heterochromatin, e.g., vertebrate heterochromatin. Heterochromatin is densely packed DNA that may be transcriptionally repressed and is often associated with the di- and tri-methylation of H3K9. In some embodiments, the heterochromatin comprises constitutive heterochromatin. In some embodiments, the heterochromatin comprises satellite sequences. In some embodiments, constitutive heterochromatin may be from human chromosomes 1, 9, 16, and/or the Y chromosome, which each contain large amounts of heterochromatin. In some embodiments, constitutive heterochromatin may be from near a centromere or telomere region of a chromosome. In some embodiments, the heterochromatin comprises facultative heterochromatin.

In some aspects, the invention provides a nucleic acid (e.g., DNA) comprising (a) one or more cognate DNA segments, e.g., one or more [(LacO)ₘ(Xaa)ₖ((TetR)ₙ] units as described above; (b) a telomeric sequence; and (c) a vertebrate origin of replication, wherein the nucleic acid does not comprise a functional native centromere or neocentromere. In some embodiments, the nucleic acid sequence comprises a portion to which cohesin binds. In some embodiments, the nucleic acid encodes modified CENP-C and/or modified CENP-T polypeptides, as described herein.

In some embodiments, a nucleic acid of the invention is linear, while in other embodiments, a nucleic acid of the invention is circular. In some embodiments, a circular nucleic acid is linearized within a cell. For example, a circular nucleic acid may comprise telomere sequences (in some embodiments at least in part in reverse orientation to one another), wherein linearization results in telomere sequences at each end of the linear molecule. In some embodiments of the invention, a nucleic acid of the invention is integrated into a chromosome or fragment thereof. Such chromosomes, and cells containing them, are aspects of the invention. In some embodiments, the chromosome or fragment thereof comprises a functional native centromere or neocentromere. In some embodiments, the chromosome or fragment thereof lacks a functional native centromere or neocentromere. In some embodiments, a nucleic acid of the invention is within a cell. Such cells are aspects of the invention.

The invention provides compositions comprising two or more modified DBCCAN polypeptide of the invention comprising a heterologous DBD. In some embodiments, a composition comprises at least 3, 4, or 5 modified CCAN polypeptides. For example, the composition could comprise at least modified CENP-C, modified CENP-T, and at least one modified CCAN selected from modified CENP-H, I, K, L, M, N, O, P, Q, R, S, U, W, and X. Optionally, the additional modified CCAN comprises a heteologous DBD. In some embodiments, a composition further comprises a DNA segment comprising recognition sequences cognate to the heterologous DBDs.

The invention provides compositions comprising (a) a modified DBCCAN polypeptide of the invention comprising a heterologous DBD; and (b) a DNA segment cognate to the heterologous DBD. The invention further provides nucleic acid-protein structures comprising (a) a modified DBCCAN of the invention comprising a heterologous DBD; and (b) a DNA segment cognate to the heterologous DBD, wherein the modified DBCCAN is bound to the DNA segment. In some embodiments, the modified DBCCAN is a modified CENP-C polypeptide. In some embodiments, the modified DBCCAN polypeptide is a modified CEPN-T polypeptide. In some embodiments, the composition or nucleic acid structure comprises at least two modified DBCCAN polypeptides, e.g., a modified CENP-C polypeptide and a modified CENP-T polypeptide, wherein each of the modified DBCCAN polypeptides comprises a heterologous DBD. These aspects of the invention encompass embodiments wherein the modified DBCCAN polypeptide, heterologous DBDs, and/or cognate DNA segments, are any of those described above. In some embodiments, the composition or nucleic acid is located in vitro (meaning “outside a cell” in this context), while in other embodiments the invention provides cells comprising the composition or nucleic acid-protein structure. In some embodiments, the cells are cultured cells, while in other embodiments the cells are within an organism, e.g., the cells (or ancestors of the cells) are introduced into or generated in the animal, or the animal is a non-human transgenic animal, e.g., a vertebrate, e.g., a rodent or avian. Transgenic animals producing one or more modified kinetochore polypeptides of the invention, wherein the genome of the animal comprises a nucleic acid encoding one or more modified kinetochore polypeptides, are an aspect of the invention. In another aspect, the invention provides a method of generating such transgenic non-human animals. In some embodiments, the transgenic animal can be generated using standard methods known in the art for generating such organisms. In some embodiments, the transgenic animal comprises an engineered kinetochore and/or artificial chromosome described herein. In some embodiments, the artificial chromosome is transmitted to progeny of the animal.

The invention further provides a multiprotein complex comprising: (a) one or more modified DBCCAN polypeptides comprising a heterologous DBD; and (b) one or more additional kinetochore polypeptides (e.g., native or modified kinetochore polypeptides in various embodiments of the invention). The complex may also be referred to as an “engineered kinetochore”. In some embodiments, the engineered kinetochore is assembled on a nucleic acid comprising a cognate DNA segment. The engineered kinetochore may be produced outside cells, e.g., by providing a composition comprising the modified DBCCAN polypeptide(s) and one or more at least partially purified additional kinetochore polypeptides or a cell lysate comprising the additional kinetochore polypeptides, wherein the composition optionally comprises a cognate DNA segment. In other embodiments, the engineered kinetochore is produced inside cells, e.g., by expressing the modified DBCCAN polypeptide(s) in cells that, in some embodiments, contain a cognate DNA segment. In some embodiments, an engineered kinetochore comprises a modified CENP-C polypeptide and a modified CENP-T polypeptide, each comprising a heterologous DBD. In some embodiments, the engineered kinetochore comprising a modified CENP-C polypeptide and a modified CENP-T polypeptide, each comprising a heterologous DBD, further comprises one or more additional kinetochore polypeptides (e.g., native or modified kinetochore polypeptides in various embodiments of the invention). In some embodiments, the one or more additional kinetochore polypeptides comprise components of the KNL-1/Mis12 complex/Ndc80 complex (KMN) network. For example, the one or more additional kinetochore components can comprise KNL1, Dsn1, and Ndc80 polypeptides. In some embodiments, the one or more additional kinetochore components comprise at least some additional outer kinetochore polypeptides, e.g., one or more additional KMN components and/or one or more members of the chromosome passenger complex (CPC). The CPC is a multiprotein complex comprising Aurora B kinase, Inner Centromere Protein (INCENP), borealin and survivin. In some embodiments, the one or more additional kinetochore components comprise at least some polypeptides of the spindle assembly checkpoint (SAC) complex, such as Mad2 (e.g., human MAD2L1 and/or MAD2L2) and/or ZW10. As known in the art, the CPC is a regulator of chromosome segregation during mitosis, which acts to correct nonbipolar microtubule-kinetochore interactions. At least in part by disrupting these interactions, the CPC is thought to create unattached kinetochores that are subsequently sensed by the spindle assembly checkpoint to prevent premature mitotic exit. In some embodiments, the one or more additional kinetochore components comprise KNL1, Dsn1, Ndc80, Zwint, Nup133, Ska1, and/or Aurora B polypeptides. See, e.g., Welburn, J, et al., (2009) The Human Kinetochore Ska1 Complex Facilitates Microtubule Depolymerization-Coupled Motility. Developmental Cell, 16(3): 374-385. In some embodiments, at least one additional kinetochore component, e.g., Bub1, ZW10 and/or CENP-E, is targeted to an engineered kinetochore. In some embodiments, such targeting is achieved by targeting additional CCAN polypeptides to the engineered kinetochore, wherein such additional CCAN polypeptides interact with the at least one additional kinetochore component, e.g., Bub1, ZW10 and/or CENP-E. In some embodiments, such targeting is achieved by modifying the additional kinetochore component, e.g., native Bub1, ZW10, and/or CENP-E, to comprise a heterologous DBD. In some embodiments, such targeting is achieved by modifying the additional kinetochore component, e.g., native Bub1, ZW10, and/or CENP-E, to comprise a moiety that binds to modified CENP-E or modified CENP-T or to a KMN component. In some embodiments, an engineered kinetochore substantially does not contain CENP-A. For example, the level of CENP-A may be less than that present in a native kinetochore by a factor of at least 10, 20, 50, or more. In some embodiments, CENP-A is undetectable (e.g., by immunostaining using anti-CENP-A antibodies). In some embodiments, satellite DNA, e.g., alpha satellite DNA, or gamma satellite DNA, is substantially absent from a site on DNA where an engineered kinetochore assembles.

In some embodiments, microtubules are associated with the KMN network components of the engineered kinetochore. In some embodiments, microtubule attachment is evidenced by deformation of a DNA comprising the engineered kinetochore assembled thereon. In some embodiments, at least one protein modification, e.g., phosphorylation, occurs in a physiologically appropriate manner. For example, Dsn may be phosphorylated at a known Aurora B phosphorylation site and/or CENP-T may be phosphorylated at a cyclin dependent kinase (CDK) phosphorylation site. In some embodiments, at least one protein modification, e.g., phosphorylation, occurs in a cell-cycle dependent manner replicating the timing with which such modification occurs at native kinetochores in proliferating cells.

The invention further provides a vertebrate artificial chromosome (VAC) comprising DNA on which an engineered kinetochore of the invention is assembled. As used herein, a vertebrate artificial chromosome is a molecule of DNA in association with proteins (e.g., histones) that behaves in a manner similar in at least some respects to naturally occurring chromosomes of a vertebrate. The DNA may be assembled with proteins to form nucleosomes. In some embodiments a VAC comprises a linear DNA molecule. In some embodiments, a VAC comprises at least 10 kB of genomic vertebrate DNA, e.g., between 10 kB and about 250 Mb in various embodiments. For example, the VAC may comprise between about 100 kB and about 50 Mb, e.g., between 1 Mb and 5 Mb, or between 5 Mb and 10 Mb, or between 10 Mb and 20 Mb, or between 20 Mb and 50 Mb, or between 50 Mb and 100 Mb of vertebrate genomic DNA in various embodiments. In some embodiments the VAC is a human artificial chromosome (HAC), wherein the HAC comprises human genomic DNA. In some embodiments, a VAC is similar to a naturally occurring chromosome in that it is located in the nucleus of a cell, is replicated during S phase of a cell cycle, contains a site at which a kinetochore (e.g., an engineered kinetochore of the invention) assembles, wherein the engineered kinetochore becomes attached to the mitotic spindle. In at least some embodiments, one copy of the replicated VAC is segregated to each daughter cell during mitosis, such that the VAC is propagated for at least 2, at least 5, at least 10 cell cycles or more. In at least some embodiments, a VAC has a high degree of mitotic stability. For example, a VAC may be correctly segregated in at least 90%, 95%, 96%, 97%, 98%, 99%, of mitoses, or more. In some embodiments, a VAC is stably transmitted over at least 5, 10, 20, 30, 40, 50, or more cell division cycles. Mitotic stability of the artificial chromosome may be measured, e.g., using fluorescence microscopy or fluorescence activated cell sorting (FACS). For example, the artificial chromosome can comprise a sequence to which a fluorescent protein binds (e.g., a modified CENP-C or CENP-T polypeptide comprising a fluorescent polypeptide domain). Other suitable methods for detecting the artificial chromosome and assessing mitotic stability could be used.

Any suitable method can be used to produce a modified kinetochore polypeptide or nucleic acid encoding it. In some embodiments, standard recombinant nucleic acid technology is used. A nucleic acid encoding the desired modified kinetochore polypeptide can be generated by nucleic acid synthesis, amplification, cloning, site-directed mutagenesis, and other methods known in the art. The nucleic acid, operably linked to appropriate expression control elements, usually in a vector such as a plasmid or virus (e.g., as part of the viral genome), is introduced into prokaryotic or eukaryotic cells. In other embodiments, a polypeptide could be produced using in vitro translation. Exemplary cells include, e.g., bacterial cells (e.g., E. coli), insect cells, mammalian cells, plant cells, fungal cells (e.g., yeast). One of skill in the art will be aware of suitable expression control elements (e.g., promoters). Promoters may be constitutive or regulatable, e.g., inducible or repressible. Exemplary promoters suitable for use in bacterial cells include, e.g., Lac, Trp, Tac, araBAD (e.g., in a pBAD vectors), phage promoters such as T7 or T3. Exemplary expression control sequences useful for directing expression in mammalian cells include, e.g., the early and late promoters of SV40, adenovirus or cytomegalovirus immediate early promoter, or viral promoter/enhancer sequences, retroviral LTRs, promoters or promoter/enhancers from mammalian genes, e.g., actin, EF-1 alpha, metallothionein, etc. The polyhedrin promoter of the baculovirus system is of use to express proteins in insect cells. One of skill in the art will be aware of numerous expression vectors that contain expression control element(s), selectable markers, cloning sites, etc., and can be conveniently used to express a polypeptide. Optionally, such vectors include sequences encoding a tag, to allow convenient production of a polypeptide comprising a tag. Suitable methods for introducing vectors into bacteria, yeast, plant, or animal cells (e.g., transformation, transfection, infection, electroporation, etc.), and, if desired, selecting cells that have taken up the vector and deriving stable cell lines. Transgenic animals or plants that express the polypeptide could be produced using methods known in the art. Cells that contain a nucleic acid encoding the modified kinetochore polypeptide can be maintained in culture for a suitable time period under conditions in which the nucleic acid is transcribed and the resulting mRNA is translated. In some embodiments, the polypeptide is isolated from cells and, optionally, further purified. In other embodiments, a modified kinetochore polypeptide could be isolated from cells or tissues of a transgenic non-human organism that expresses it. Standard protein isolation/purification techniques can be used. In some embodiments, affinity-based methods are used. For example, an antibody to the polypeptide can be employed. In the case of a polypeptide that comprises a tag, an appropriate isolation method can be selected depending on the particular tag. Vectors include, e.g., plasmids, P1-derived artificial chromosomes (PACs), bacterial artificial chromosomes (BACs), yeast artificial chromosomes (YACs), and virus vectors. In some embodiments, a vector capable of containing a large nucleic acid (e.g., at least 10 kB, 100 kB, 1 Mb, or more) is used. Exemplary virus vectors include herpesvirus family vectors, e.g., herpes simplex virus, herpesvirus saimiri virus, Epstein Barr virus, human cytomegalovirus. In some embodiments a virus is a replication-defective virus. The invention provides such vectors, containing one or more nucleic acids of the invention. In some embodiments, a nucleic acid is packaged in a virion.

In some embodiments, a nucleic acid or artificial chromosome is assembled by recombining various nucleic acids outside or within cells. A variety of methods are available. For example, a yeast, viral, phage, or bacterial recombinase can be used, such as Cre recombinase, which mediates recombination at LoxP sites, or the yeast Frt/Flp system or a viral integrase, or a transposase, or a bacterial invasin. An artificial chromosome may be assembled within a cell. In some embodiments, multiple nucleic acids are introduced into a cell, e.g., one or more nucleic acids that collectively comprise cognate DBDs, DNA comprising one or more genes of interest, and telomere sequences. Recombination occurring within the cell can generate a nucleic acid comprising elements needed for propagation of the nucleic acid as an artificial chromosome. For example, in some embodiments, recombination can result in a linear molecule comprising cognate DNA segment, at least one vertebrate origin or replication, and having telomere sequences at each end. In some embodiments, at least one of the nucleic acids comprises a gene encoding a selectable marker (e.g., a drug resistance or nutritional marker) or detectable marker. In some embodiments, multiple nucleic acids, each comprising a different marker gene, are introduced. In some embodiments, hypoxanthine aminopterin thymidine (HAT) selection is sued. Cells in which the nucleic acids assemble to form a heritable chromosome (or progeny thereof) are positive for all of the markers and can be identified or selected (e.g., by culturing the cells in selective medium). In certain embodiments of the invention it is contemplated to use applicable methods for chromosome assembly and/or delivery, e.g., for “bottom-up” generation of human artificial chromosomes described in Grimes, B R and Monaco, Z L, Artificial and engineered chromosomes: developments and prospects for gene therapy (2005) Chromosoma, 114:230-241. In some embodiments, a “backbone” nucleic acid is provided, and nucleic acids that comprise cognate DBDs, telomere sequences, that encode one or more modified DBCCANs, and/or comprise a gene of interest are integrated into the backbone sequence.

In some embodiments, a nucleic acid or artificial chromosome of the invention is delivered to a cell using cell fusion or microcell-mediated chromosome transfer (MMCT). In some embodiments, a chromosome is delivered as described in Ikeno, M., et al. (2009) Manipulating transgenes using a chromosome vector. Nucleic Acids Res. 37(6):e44. In some embodiments, a nucleic acid or artificial chromosome of the invention is delivered to a cell using or assisted by a lipid-based delivery composition, ultrasound, electroporation and/or microparticle bombardment.

A wide variety of cell types can be used in embodiments of the inventive methods and compositions. Cells and cell lines that contain or express an inventive modified kinetochore polypeptide or nucleic acid, engineered kinetochore, or artificial chromosome are aspects of the invention. A cell could originate from any organism of interest, e.g., a vertebrate, e.g., a mammal or avian. In some embodiments, a cell is a primate cell, e.g., a human cell. A cell could be a primary cell, immortalized cell, cancer cell, etc. Often, a cell is a member of a population of cells which is composed of cells that are substantially genetically identical, e.g., a cell line. A cell line can be descended from a single cell or from multiple cells isolated from a single individual. A cell can originate from a tissue or organ of interest or can have a property of interest. In some embodiments, a cell is an epithelial cell, fibroblast, kidney cell, rhabdosarcoma or rhabdomyosarcoma, lung, or bronchial cell, pre-adipocyte, or adipocyte. In some embodiments a cell originates from breast, bladder, bone, brain, bronchus, cervix, colon, endometrium, esophagus, larynx, liver, lung, nerve, muscle, ovary, pancreas, prostate, stomach, kidney, skin, testis, or thyroid gland. Numerous cell lines are known in the art, many of which can be obtained from repositories such as the American Type Culture Collection, Coriell Cell Repositories, European Collection of Cell Cultures, Japanese Collection of Research Bioresources, or from a variety of commercial suppliers. In some embodiments, a cell is a Chinese hamster ovary (CHO) cell. In some embodiments, a cell is a COS cell, e.g., a COS-1 or COS-7 cell. In some embodiments, a cell is a HeLa cell. In some embodiments, a cell is a Vero, RD, CHO, HEK-293, HMEC, MDCK, NIH-3T3, HEp-2, A549, or BEAS-2B cell. In some embodiments, a cell is a tumor cell. In some embodiments a tumor cell originates from a carcinoma. In some embodiments a tumor cell originates from a sarcoma, e.g., a fibrosarcoma. In some embodiments a tumor cell originates from a hematologic malignancy, e.g., a lymphoma or leukemia or myeloma. In some embodiments a tumor cell originates from a breast, bladder, bone, brain, cervical, colon, endometrial, esophageal, head and neck, laryngeal, liver, lung (small cell or non-small cell), ovarian, pancreatic, prostate, stomach, renal, skin (e.g., basal cell, melanoma, squamous cell), testicular, or thyroid cancer. The tumor cell may be a cell of an established tumor cell line (e.g., one of the NCI-60 tumor cell lines) or another tumor cell line known in the art or newly established.

In some embodiments, a cell is a stem cell. In some embodiments, the stem cell is an embryonic stem (ES) cell, e.g., a human or mouse ES cell. In some embodiments, the cell is an induced pluriopotent stem (iPS) cell, e.g., a human or mouse iPS cell. As known in the art, a wide variety of somatic cell types can be reprogrammed in vitro to a pluripotent state, e.g., through introduction of various combinations of transcription factors, e.g., the four transcription factors Oct4, Sox2, Klf4, and c-Myc (with c-Myc being dispensable, although omitting c-Myc reduced reprogramming efficiency), or the four transcription factors Oct4, Nanog, Sox2, and Lin28 (see, e.g., Meissner, A., et al., Nat. Biotechnol., 25(10):1177-81 (2007); Yu, J., et al, Science, 318(5858):1917-20 (2007); and Nakagawa, M., et al., Nat. Biotechnol., 26(1):101-6 (2008). Such transcription factors are often referred to as “reprogramming factors”). A variety of approaches are available to geneate iPS cells.

In some embodiments, a cell is a hematopoietic stem cell. In some embodiments a cell is a neural stem cell. In some embodiments a cell is a myoblast.

In some embodiments, a cell used in a method described herein is one that has been used in the art to generate or maintain human artificial chromosomes. Exemplary cell types are human HT1080 cells and chicken DT40 cells.

### III. Kits

The invention further provides kits comprising one or more of any of the inventive nucleic acids, polypeptides, cells, compositions, and/or reagents suitable for performing any of the inventive methods. For example, a kit may contain a nucleic acid encoding a modified CENP-C and/or modified CENP-T polypeptide comprising a heterologous DBD, wherein the coding sequence is operably linked to expression control elements. The nucleic acid may further cognate DNA segment(s) and/or the kit may further comprise a nucleic acid comprising cognate DNA segment(s). The kit may contain one or more antibodies useful to detect a modified or native kinetochore polypeptide. The contents of a kit can be packaged in multiple in containers, each of which contains one or more components of the kit, and which may be provided within a larger container. The individual containers of the kit may be maintained in close confinement for commercial sale. A kit can contain instructions for using the contents to perform any of the methods or make any of the compositions of the invention.

### IV. Compositions and Methods for Identifying Compounds

The invention provides methods of identifying compounds that modulate kinetochore assembly and/or function. Further provided are compositions useful for performing the inventive methods. In some aspects, the invention provides a method of identifying a compound that modulates modulate kinetochore assembly and/or function, the method comprising (a) providing a cell comprising components of an engineered kinetochore of the invention; (b) contacting the cell with a test compound; (c) determining whether the test compound modulates (e.g., inhibits, increases, or otherwise affects) the assembly or function of an engineered kinetochore. In some embodiments, a method is used to identify compounds that interfere with kinetochore assembly and/or with one or more kinetochore functions. Such compounds may be useful, e.g., to inhibit cell division, e.g., for treatment of disorders involving excessive cell proliferation, such as tumors. In some embodiments, a method is used to identify compounds that promote engineered kinetochore assembly and/or function. Such compounds may be used, e.g., to improve methods of using the engineered kinetochores and/or artificial chromosomes of the invention.

The invention further provides methods of identifying compounds that modulate structure, maintenance, modification, and/or activity of a nucleic acid (e.g., a DNA) or nucleic acid-associated protein. Further provided are compositions useful for performing the inventive methods. In some aspects, the invention provides a method of identifying a compound that modulates structure, maintenance, modification, and/or activity of a nucleic acid (e.g., a DNA) the method comprising (a) providing a cell comprising an artificial chromosome of the invention, wherein the artificial chromosome comprises the nucleic acid; (b) contacting the cell with a test compound; (c) determining whether the test compound modulates structure, maintenance, modification, and/or activity of the nucleic acid. The nucleic acid may be isolated from the cell and further analyzed (e.g., to assess its modification state) For example, an inventive method may be used to identify compounds that affect DNA or histone modifications such as methylation, acetylation, etc. Compounds identified using an inventive method may be used, e.g., to alter gene expression (e.g., to promote or inhibit transcriptional silencing of particular genes), which could be used to treat disorders involving aberrant gene expression, for example.

The invention further provides methods of identifying compounds useful for modulating expression or activity of a gene product of interest. Further provided are compositions useful for performing the inventive methods. In some aspects, the invention provides a method of identifying a compound that modulates expression or activity of a gene product, the method comprising (a) providing a cell comprising an artificial chromosome of the invention, wherein the artificial chromosome comprises a gene that encodes the gene product; (b) contacting the cell with a test compound; (c) determining whether the test compound affects (e.g., inhibits, increases) the expression or activity of the gene product. Compounds identified using an inventive method may be used for any purpose in which it is desired to alter expression or activity of the gene product. In some embodiments, a compound is useful for increasing production of a functional gene product of interest by cells. In some embodiments, the cells are isolated cells. In some embodiments, a compound is useful for increasing or decreasing production of a gene product in vivo.

A compound identified using an inventive method, e.g., a compound identified as as a modulator of a DNA or gene product of interest, can be tested in cell culture or in animal models (“in vivo”) to further characterize its effects. Cytotoxicity can be assessed e.g., using any of a variety of assays for cell viability and/or proliferation such as a cell membrane integrity assay, a cellular ATP-based viability assay, a mitochondrial reductase activity assay, a BrdU, EdU, or H3-Thymidine incorporation assay, a DNA content assay using a nucleic acid dye, such as Hoechst Dye, DAPI, Actinomycin D, 7-aminoactinomycin D or propidium iodide, a cellular metabolism assay such as AlamarBlue, MTT, XTT, and CellTitre Glo, etc. The compound can be tested in an animal model of a disorder, e.g., a genetic disorder.

One of skill in the art would be aware of suitable methods to assess expression and/or activity of a gene product of interest. Methods known in the art can be used for measuring mRNA or protein. A variety of different hybridization-based or amplification-based methods are available to measure RNA. Examples include Northern blots, microarray (e.g., oligonucleotide or cDNA microarray), reverse transcription (RT)-PCR (e.g., quantitative RT-PCR), or reverse transcription followed by sequencing. The TaqMan® assay and the SYBR® Green PCR assay are commonly used real-time PCR techniques. Other assays include the Standardized (Sta) RT-PCR™ (Gene Express, Inc., Toledo, Ohio) and QuantiGene® (Panomics, Inc., Fremont, Calif.). In some embodiments the level of mRNA is measured. In other embodiments, a reporter-based system is used. Assays for activity of a gene product (e.g., enzymatic activity, binding activity) would be selected base on the particular activity of interest. In general, assays could be cell-free or cell-based in various embodiments of the invention.

A wide variety of test compounds can be used in the inventive methods. For example, a test compound can be a small molecule, polypeptide, peptide, nucleic acid, oligonucleotide, lipid, carbohydrate, or hybrid molecule. Compounds can be obtained from natural sources or produced synthetically. Compounds can be at least partially pure or may be present in extracts or other types of mixtures. Extracts or fractions thereof can be produced from, e.g., plants, animals, microorganisms, marine organisms, fermentation broths (e.g., soil, bacterial or fungal fermentation broths), etc. In some embodiments, a compound collection (“library”) is tested. The library may comprise, e.g., between 100 and 500,000 compounds, or more. Compounds are often arrayed in multwell plates. They can be dissolved in a solvent (e.g., DMSO) or provided in dry form, e.g., as a powder or solid. Collections of synthetic, semi-synthetic, and/or naturally occurring compounds can be tested. Compound libraries can comprise structurally related, structurally diverse, or structurally unrelated compounds. Compounds may be artificial (having a structure invented by man and not found in nature) or naturally occurring. In some embodiments, a library comprises at least some compounds that have been identified as “hits” or “leads” in other drug discovery programs and/or derivatives thereof. A compound library can comprise natural products and/or compounds generated using non-directed or directed synthetic organic chemistry. Often a compound library is a small molecule library. Other libraries of interest include peptide or peptoid libraries, cDNA libraries, and oligonucleotide libraries. A library can be focused (e.g., composed primarily of compounds having the same core structure, derived from the same precursor, or having at least one biochemical activity in common).

Compound libraries are available from a number of commercial vendors such as Tocris BioScience, Nanosyn, BioFocus, and from government entities. For example, the Molecular Libraries Small Molecule Repository (MLSMR), a component of the U.S. National Institutes of Health (NIH) Molecular Libraries Program is designed to identify, acquire, maintain, and distribute a collection of >300,000 chemically diverse compounds with known and unknown biological activities for use, e.g., in high-throughput screening (HTS) assays (see https://mli.nih.gov/mli/). The NIH Clinical Collection (NCC) is a plated array of approximately 450 small molecules that have a history of use in human clinical trials. These compounds are highly drug-like with known safety profiles. The NCC collection is arrayed in six 96-well plates. 50 μl of each compound is supplied, as an approximately 10 mM solution in 100% DMSO. In some embodiments, a collection of compounds comprising “approved human drugs” is tested. An “approved human drug” is a compound that has been approved for use in treating humans by a government regulatory agency such as the US Food and Drug Administration, European Medicines Evaluation Agency, or a similar agency responsible for evaluating at least the safety of therapeutic agents prior to allowing them to be marketed. The test compound may be, e.g., an antineoplastic, antibacterial, antiviral, antifungal, antiprotozoal, antiparasitic, antidepressant, antipsychotic, anesthetic, antianginal, antihypertensive, antiarrhythmic, antiinflammatory, analgesic, antithrombotic, antiemetic, immunomodulator, antidiabetic, lipid- or cholesterol-lowering (e.g., statin), anticonvulsant, anticoagulant, antianxiety, hypnotic (sleep-inducing), hormonal, or anti-hormonal drug, etc. In some embodiments, a compound is one that has undergone at least some preclinical or clinical development or has been determined or predicted to have “drug-like” properties. For example, the test compound may have completed a Phase I trial or at least a preclinical study in non-human animals and shown evidence of safety and tolerability. In some embodiments, a test compound is substantially non-toxic to cells of an organism to which the compound may be administered or cells in which the compound may be tested, at the concentration to be used or, in some embodiments, at concentrations up to 10-fold, 100-fold, or 1.000-fold higher than the concentration to be used. For example, there may be no statistically significant effect on cell viability and/or proliferation, or the reduction in viability or proliferation can be no more than 1%, 5%, or 10% in various embodiments. Cytotoxicity and/or effect on cell proliferation can be assessed using any of a variety of assays (some of which are mentioned above). In some embodiments, a test compound is not a compound that is found in a cell culture medium known or used in the art, e.g., culture medium suitable for culturing vertebrate, e.g., mammalian cells or, if the test compound is a compound that is found in a cell culture medium known or used in the art, the test compound is used at a different, e.g., higher, concentration when used in a method of the present invention.

In some embodiments, a method of identifying compounds is performed using a high throughput screen (HTS). A high throughput screen can utilize cell-free or cell-based assays. High throughput screens often involve testing large numbers of compounds with high efficiency, e.g., in parallel. For example, tens or hundreds of thousands of compounds can be routinely screened in short periods of time, e.g, hours to days. Often such screening is performed in multiwell plates containing, e.g., e.g., 96, 384, 1536, 3456, or more wells (sometimes referred to as microwell or microtiter plates or dishes) or other vessels in which multiple physically separated cavities are present in a substrate. High throughput screens can involve use of automation, e.g., for liquid handling, imaging, data acquisition and processing, etc. Without limiting the invention in any way, certain general principles and techniques that may be applied in embodiments of a HTS of the present invention are described in Macarrón R & Hertzberg R P. Design and implementation of high-throughput screening assays. Methods Mol. Biol., 565:1-32, 2009 and/or An W F & Tolliday N J., Introduction: cell-based assays for high-throughput screening. Methods Mol. Biol. 486:1-12, 2009, and/or references in either of these. Exemplary methods are also disclosed in High Throughput Screening: Methods and Protocols (Methods in Molecular Biology) by William P. Janzen (2002) and High-Throughput Screening in Drug Discovery (Methods and Principles in Medicinal Chemistry) (2006) by Jorg Hiser.

### V. Selected Applications and Compositions

In some aspects, the invention provides a method of maintaining a selected DNA sequence in a eukaryotic cell, the method comprising: (a) providing a eukaryotic cell that produces a modified CENP-C polypeptide and a modified CENP-T polypeptide, wherein the modified CENP-C and modified CENP-T polypeptides each comprise a heterologous DNA binding domain, and wherein the cell comprises an artificial chromosome that comprises (i) a DNA segment cognate to the heterologous DNA binding domain of the modified CENP-C polypeptide, (ii) a DNA segment heterologous to the DNA binding domain of the modified CENP-T polypeptide, and (iii) a selected DNA sequence; and (b) maintaining the cell under conditions suitable for binding of the modified CENP-C and modified CENP-T polypeptides to their cognate DNA segments. In some embodiments, the eukaryotic cell is a vertebrate cell, which is optionally a mammalian or avian cell, e.g., a human cell. In some embodiments, the selected DNA sequence encodes a functional RNA or precursor thereof, e.g., a microRNA, short interfering RNA, short hairpin RNA, long non-coding RNA, transfer RNA, ribosomal RNA, antisense RNA, or ribozyme. In some embodiments the selected DNA sequence encodes a polypeptide. In some embodiments, the cell is maintained in culture, e.g., under conditions suitable for cell survival and, in some embodiments, cell division. In some embodiments, the artificial chromosome is duplicated during the S phase of the cell cycle and one of the two resulting chromosomes is transmitted to each daughter cell. In some embodiments, a product of interest is DNA, e.g., DNA contained in an artificial chromosome. For example, an artificial chromosome or portion thereof can be isolated from the cell and, optionally purified, e.g., using gel electrophoresis or other suitable methods.

In some aspects, the invention provides a method of producing a gene product in a eukaryotic cell, the method comprising: (a) providing a eukaryotic cell that produces a modified CENP-C polypeptide and a modified CENP-T polypeptide, wherein the modified CENP-C and modified CENP-T polypeptides each comprise a heterologous DNA binding domain, and wherein the cell comprises an artificial chromosome that comprises (i) a DNA segment cognate to the heterologous DNA binding domain of the modified CENP-C polypeptide, (ii) a DNA segment heterologous to the DNA binding domain of the modified CENP-T polypeptide, and (iii) a DNA segment that encodes the gene product, wherein the sequence encoding the gene product is operably linked to expression control elements capable of directing expression of the sequence encoding the gene product in the cell; and (b) maintaining the cell under conditions in which the gene is expressed. In some embodiments, the gene product is a functional RNA or precursor thereof, e.g., a microRNA, short interfering RNA, short hairpin RNA, long non-coding RNA, transfer RNA, ribosomal RNA, antisense RNA, or ribozyme. In some embodiments the gene product is a polypeptide. The polypeptide can be any polypeptide, e.g., an enzyme, hormone, growth factor, ECM polypeptide, therapeutically or industrially useful polypeptide. In some embodiments, the polypeptide is one that is encoded by an extremely large gene, such as Titin.

In some aspects, the invention provides methods of treatment and compositions, e.g., pharmaceutical compositions, of use in the methods. In some aspects, the invention provides a method of treating a subject in need of treatment for a disorder, wherein the method comprises administering a cell comprising administering a gene product (e.g., a protein) produced by a cell that contains a VAC of the invention to the subject, wherein the VAC comprises a gene that encodes the gene product. In some aspects, the invention provides a method of treating a subject in need of treatment for a disorder, wherein the method comprises administering a cell comprising a VAC of the invention to the subject. In some embodiments, the method comprises delivering a VAC, e.g., a HAC, to at least some cells of the subject. In some embodiments, the VAC is delivered by administering one or more nucleic acids to the subject, wherein such delivery results in assembly or introduction of the VAC in the nucleus of at least some cells of the subject. In some embodiments, the nucleic acids are delivered in a suitable vector. In some embodiments the method comprises administering a compound identified as described herein to a subject. “Administration” can comprise direct administration or indirect administration. “Indirect” administration comprises activities such as providing, prescribing, directing another individual to administer, or in any way making a composition available to a subject.

The invention contemplates treatment of a variety of disorders in human and/or animal subjects. In some embodiments, the disorder is one for which an effective pharmacological therapy (e.g., small molecule or polypeptide) does not exist, is not in commercial use, or is not widely used. In some aspects, the invention provides methods of treating a disorder resulting from mutation or altered (e.g., decreased) expression of a gene. Exemplary diseases and conditions include, e.g., muscular dystrophy (e.g., Duchenne muscular dystrophy), cystic fibrosis, retinitis pigmentosa, immunodeficiencies, inherited skin adhesion disorders such as epidermolysis bullosa, and enzyme deficiencies. In some embodiments, a deficiency of a protein is ameliorated by administering an artificial chromosome of the invention comprising a gene encoding the protein, e.g., the artificial chromosome comprises a genomic DNA comprising the gene. In some embodiments a gene encodes a structural protein such as a collagen. In some embodiments, a gene encodes an enzyme. In another embodiment, deficiency of a tumor suppressor gene (e.g., the retinoblastoma gene or Wilms tumor suppressor gene) is ameliorated by administering an artificial chromosome of the invention comprising the tumor suppressor gene. In some embodiments, the HAC comprises a gene whose gene product inhibits expression or activity of a molecule (e.g., a polypeptide) that is at least in part responsible for a disorder. For example, the gene may encode a short hairpin RNA, microRNA precursor, antisense RNA, or other RNA molecule capable of interfering with gene expression, or a dominant negative version of a polypeptide. In some embodiments a HAC of the invention is used to treat any disorder whose treatment using gene therapy is contemplated.

In some aspects, the invention contemplates ex vivo uses of an artificial chromosome of the invention. For example, cells intended for use in transplantation (e.g., xenotransplantation or transplantation into an individual of the same species) can be contacted ex vivo with nucleic acids sufficient to cause the cells to contain an artificial chromosome of the invention, wherein the artificial chromosome comprises a gene of interest. The cells are subsequently administered to the subject. In some embodiments, the cells contribute to repair or replacement of a damaged or diseased tissue or organ. Any methods known in the art for administering cell-based therapy can be used in various embodiments of the invention. For example, cells can be administered with matrices or scaffolds. Cells can be administered locally at a site of tissue damage or loss and/or wherever their engraftment is desired, e.g., in the liver, bone marrow, etc. In some embodiments, autologous cells or at least partly genetically matched cells, e.g., histocompatible cells, are used. For example, cells can be removed from a subject, treated ex vivo to introduce an artificial chromosome of the invention, optionally expanded ex vivo, and then administered to the subject. Cells can be prepared in an acceptable manner for administration to a subject, e.g., they can be free or substantially free of human pathogens and substances that would be harmful if administered to a human.

Inventive methods of treatment can include a step of identifying a subject suffering from or at risk of a disorder, a step of producing a subject-specific therapeutic agent of the invention, and/or a step of prescribing, providing, or administering a composition of the invention to the subject.

Any of a variety of methods may be employed to identify a subject in need of treatment according to the present invention. For example, such methods include clinical diagnosis based at least in part on symptoms, medical history (if available), physical examination, laboratory tests, imaging studies, immunodiagnostic assays, nucleic acid based diagnostics, and/or histopathologic analysis of an appropriate sample obtained from the subject.

The compositions disclosed herein and/or identified using a method described herein may be administered by any suitable means such as orally, intranasally, subcutaneously, intramuscularly, intravenously, intra-arterially, parenterally, intraperitoncally, intrathecally, intratracheally, ocularly, sublingually, vaginally, rectally, dermally, or by inhalation, e.g., as an aerosol. Depending upon the type of disorder to be treated, compositions of the invention may, for example, be inhaled, ingested or administered by systemic routes. Thus, a variety of administration modes, or routes, are available. The particular mode selected will depend, of course, upon the particular compound selected, the particular condition being treated and the dosage required for therapeutic efficacy. The methods of this invention, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. The term “parenteral” includes intravenous, intramuscular, intraperitoneal, subcutaneous, intraosseus, and intrasternal injection, or infusion techniques. In some embodiments, a route of administration is parenteral or oral. Optionally, a route or location of administration is selected based at least in part on the particular viral infection and/or location of infected tissue. For example, a compound may be delivered to or near an affected tissue or a tissue in which a gene is normally expressed.

Suitable preparations, e.g., substantially pure preparations, of a compound may be combined with one or more pharmaceutically acceptable carriers or excipients, etc., to produce an appropriate pharmaceutical composition. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds is well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21 st Edition. Philadelphia, Pa. Lippincott Williams & Wilkins, 2005, for additional discussion of pharmaceutically acceptable substances and methods of preparing pharmaceutical compositions of various types. which are incorporated herein by reference in their entirety).

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate, fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic. Pharmaceutical compositions and compounds for use in such compositions may be manufactured under conditions that meet standards or criteria prescribed by a regulatory agency. For example, such compositions and compounds may be manufactured according to Good Manufacturing Practices (GMP) and/or subjected to quality control procedures appropriate for pharmaceutical agents to be administered to humans.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art.

Formulations for oral delivery may incorporate agents to improve stability in the gastrointestinal tract and/or to enhance absorption.

For administration by inhalation, inventive compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The present invention also contemplates delivery of compositions using a nasal spray or other forms of nasal administration.

For topical applications, pharmaceutical compositions may be formulated in a suitable ointment, lotion, gel, or cream containing the active components suspended or dissolved in one or more pharmaceutically acceptable carriers suitable for use in such composition.

For local delivery to the eye, the pharmaceutically acceptable compositions may be formulated as solutions or micronized suspensions in isotonic, pH adjusted sterile saline, e.g., for use in eye drops, intravitreal injection, or in an ointment.

Pharmaceutical compositions may be formulated for transmucosal or transdermal delivery. For transmucosal or transdermal administration, penetrants appropriate to the barrier to be permeated may be used in the formulation. Such penetrants are generally known in the art. Inventive pharmaceutical compositions may be formulated as suppositories (e.g., with conventional suppository bases such as cocoa butter and other glycerides) or as retention enemas for rectal delivery.

In some embodiments, a pharmaceutical composition includes one or more agents intended to protect the active agent(s) against rapid elimination from the body, such as a controlled release formulation, implants, microencapsulated delivery system, etc. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle-based delivery systems are known in the art for delivery of nucleic acids. The invention contemplates use of such compositions. Liposomes or other lipid-based particles can also be used.

Compositions of the invention, when administered to a subject, are preferably administered for a time and in an amount sufficient to treat the disorder for which they are administered. Therapeutic efficacy and/or toxicity can be assessed by standard pharmaceutical procedures in cell cultures or experimental animals. The data obtained from cell culture assays and animal studies can be used in formulating a range of dosages suitable for use in humans or other subjects. Different doses for human administration can be further tested in clinical trials in humans as known in the art. The dose used may be the maximum tolerated dose or a lower dose. In some embodiments a single dose is administered while in other embodiments multiple doses are administered. The specific dose level for a subject may depend upon a variety of factors including the activity of the specific agent(s) employed, severity of the disease or disorder, the age, body weight, general health of the subject, etc.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

### CENP-A Mis-Targeting Results in the Ectopic Recruitment of a Small Subset of Kinetochore Proteins

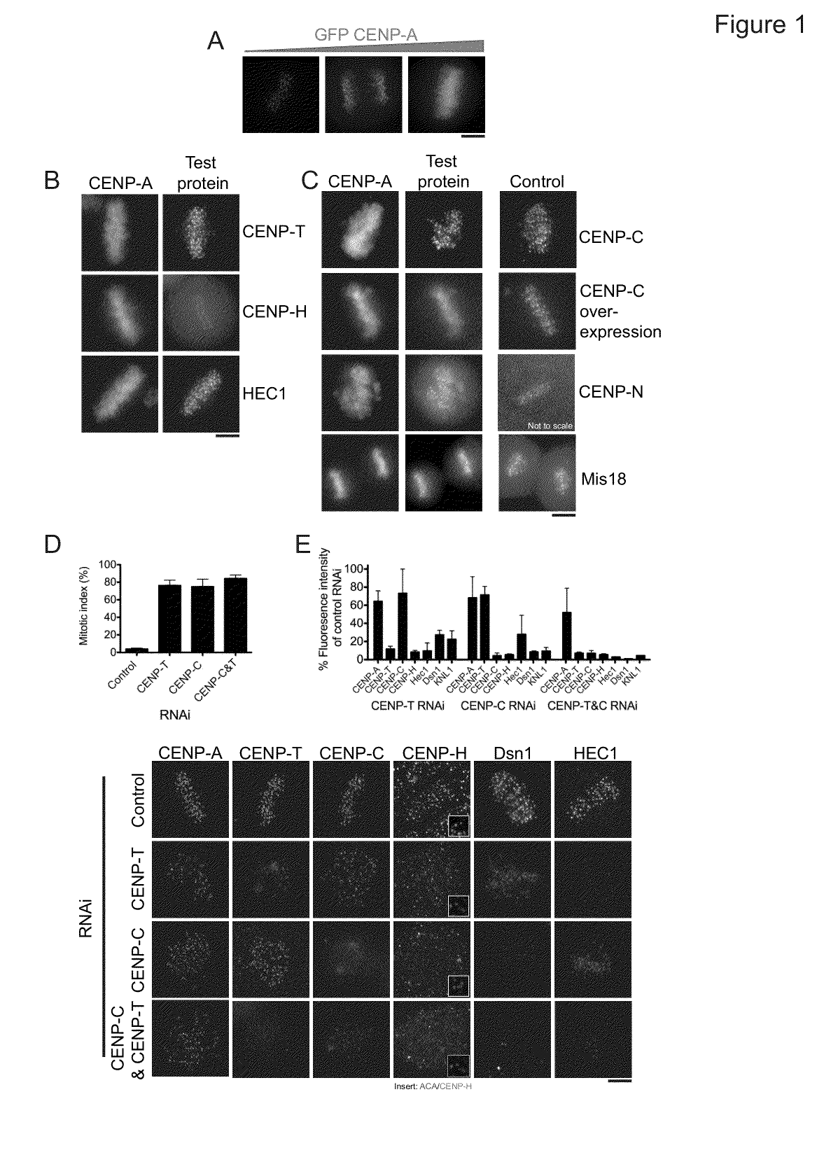

Previous work of others has suggested that CENP-A is required for accurate chromosome segregation in all organisms where is it present, as loss of CENP-A leads to a failure of kinetochore formation (Black et al., 2007; Howman et al., 2000; Oegema et al., 2001). However, in human cells, the molecular role of CENP-A in kinetochore assembly has remained unclear. Over-expression of CENP-A in human cells leads to its incorporation into non-centromeric chromatin (FIG. 1A) (Van Hooser et al., 2001). To analyze the role of CENP-A for kinetochore assembly, we carried out a comprehensive analysis of the consequences of CENP-A over-expression. Moderate over-expression of GFP tagged CENP-A in HeLa cells had no discernable effect on chromosome segregation, and we were able to generate stable cell lines with GFP-CENP-A present on chromosomes arms (data not shown). To determine the role of CENP-A in kinetochore protein localization, we monitored the localization of a panel of inner and outer kinetochore proteins in the presence of either over-expressed fluorescently tagged CENP-A or histone-H2B. All kinetochore proteins tested localized normally to centromere foci in the presence of GFP-H2B (data not shown). The majority of tested kinetochore proteins, including CENP-T, CENP-H, CENP-O, Ndc80/Hec1, Dsn1, KNL1, Aurora B, INCENP, MCAK, Bub1, BubR1, CENP-E, Nup133, and ZW10 were restricted to centromeres in the presence of CENP-A mis-targeting to chromosome arms (FIG. 1B; data not shown).

However, three kinetochore proteins—CENP-C, CENP-N, and Mis18—were mis-localized to chromosome arms by CENP-A over-expression (FIG. 1C). Antibody staining revealed that endogenous CENP-C was not mis-localized by CENP-A over-expression, in contrast to previous observations (Van Hooser et al., 2001). However, co-over-expression of CENP-C along with CENP-A resulted in its mis-localization to chromosome arms. This is consistent with published in vitro data (Carroll et al., 2010), but suggests that an interaction with CENP-A is not the only mechanism by which CENP-C is recruited to centromeres. The mis-localization of CENP-N by CENP-A over-expression is also consistent with in vitro binding assays, in which CENP-N has been shown to possess direct CENP-A binding activity (Carroll et al., 2009). In addition to mis-localization, we also observed a general increase in CENP-N levels during mitosis in cells over-expressing CENP-A, as endogenous CENP-N was barely detectable during mitosis in controls cells. Mis18 has been suggested to act as a CENP-A loading factor (Fujita et al., 2007; Hayashi et al., 2004; Maddox et al., 2007), and is present at kinetochores only during late anaphase/telophase. We found that the over-expression of CENP-A did not affect the timing of Mis18 localization to chromosomes (data not shown) but did target Mis18 to chromosome arms in anaphase/telophase (FIG. 1C).

Taken together, these data indicate that while CENP-A is essential for kinetochore formation, it is not sufficient for kinetochore assembly in human cells as only three of the fifteen kinetochore components tested were recruited to ectopic CENP-A sites. This contrasts with results observed in Drosophila, where over-expression of CENP-A is sufficient for ectopic kinetochore formation (Heun et al., 2006).

### Example 2

### CENP-T/W and CENP-C are Essential for Kinetochore Assembly

One possible explanation for the difference in the sufficiency of CENP-A for kinetochore assembly in Drosophila and human cells is the presence of additional chromatin proximal kinetochore proteins in human cells. With the exception of CENP-C, the constitutive centromere associated CCAN proteins are absent from Drosophila. To address this possibility, we next analyzed the requirements of other chromatin proximal kinetochore proteins for kinetochore assembly. Both CENP-C and CENP-T/W have been shown to have DNA-binding properties (Hori et al., 2008; Sugimoto et al., 1994; Yang et al., 1996). Depletion of CENP-C or CENP-T alone or in combination led to severe defects in chromosome alignment and a mitotic arrest (FIG. 1D) (Hori et al., 2008; Kalitsis et al., 1998). However, CENP-C and CENP-T co-depletion caused only a mild reduction in CENP-A levels at centromeres (FIG. 1E), confirming that CENP-A functions upstream of CENP-C and CENP-T in the kinetochore assembly hierarchy. Similarly, depletion of CENP-C or CENP-T individually caused only a mild reduction in the levels of the reciprocal protein at kinetochores, suggesting these two DNA-binding proteins are recruited independently to kinetochores. In contrast, simultaneous depletion of CENP-C and CENP-T prevented the kinetochore localization of all other proteins tested, including components of the KMN network (FIG. 1E). Individual depletion of CENP-C or CENP-T caused a severe reduction in CENP-H levels at kinetochores, as well as a reduction in components of the KMN network. However, following CENP-T depletion, the levels of Ndc80/Ndc80/Hec1 at kinetochores were reduced to below 10%, while Dsn1 or KNL1 remained at ˜20% of control levels. In contrast, following CENP-C depletion, the levels of Dsn1 and KNL1 were reduced to below 10%, while Ndc80/Hec1 remained close to 30% of control levels (FIG. 1E). Thus, while CENP-C and CENP-T are both required for full kinetochore assembly, they display potential differences in their requirement for the recruitment of KMN network components.

### Example 3

### Sequence Independent DNA Binding of the CENP-T/W Complex is Required for Kinetochore Formation

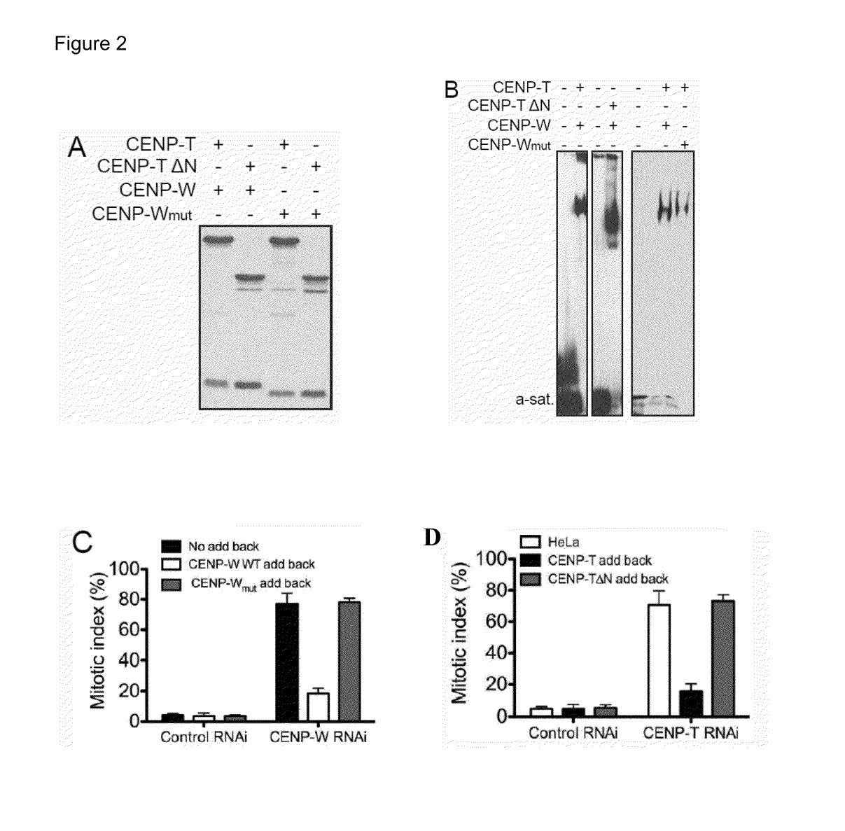

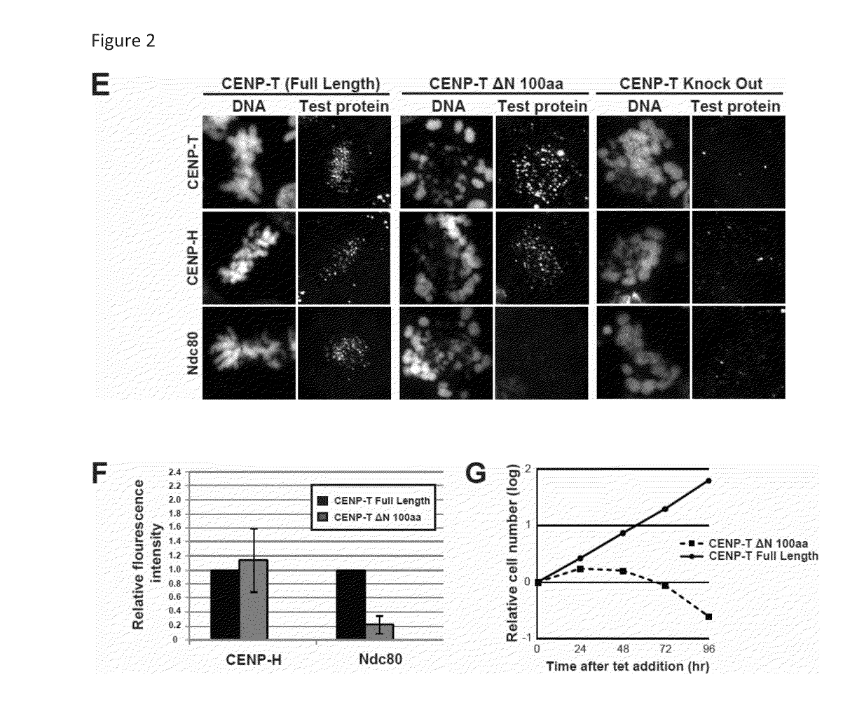

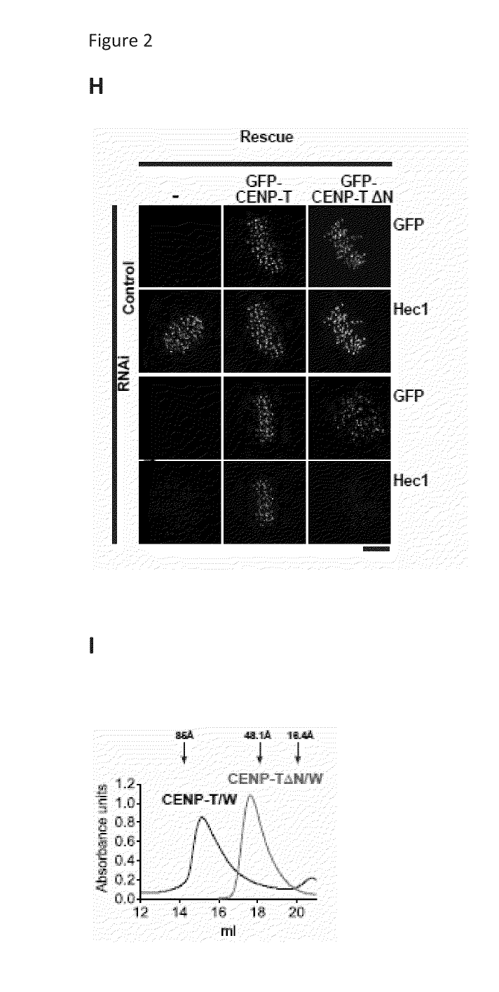

The CENP-T/W complex is crucial for correct assembly of the kinetochore. As CENP-A is not sufficient to specify CENP-T/W localization, it remains unclear how this essential complex is targeted to centromeres. To investigate the mechanism of centromeric targeting of the CENP-T/W complex, we analyzed its DNA binding properties using bacterially expressed recombinant CENP-T/W complex (FIG. 2A). The CENP-T/W complex displayed direct DNA binding activity in vitro, as indicated by retardation of labeled alpha-satellite DNA in an Electro-Mobility Shift Assay (EMSA) (FIG. 2B). We previously identified several residues within CENP-W as critical for DNA binding in chicken DT40 cells (Hori et al., 2008). These residues (R19, K23, R24, R34, R87) were also critical for CENP-W function in human cells. Depletion of CENP-W by RNAi in HeLa cells caused a strong mitotic arrest. This phenotype could be rescued by expression of RNAi resistant wild-type GFP-CENP-W (FIG. 2C). However, expression of GFP-CENP-W with the five critical R/K residues mutated to alanine (referred to as Wₘᵤₜ), was unable to rescue the mitotic arrest caused by CENP-W depletion. Consistent with a defect in DNA binding, GFP-CENP-Wₘᵤₜ did not localize to centromeres in interphase or mitosis (data not shown). Mutation of these residues in recombinant CENP-W did not alter its binding to CENP-T (FIG. 2A). However, in vitro binding of CENP-T/Wₘᵤₜ to alpha-satellite DNA sequences was reduced compared to the wild-type complex (FIG. 2B). The residual DNA binding activity is likely due to the contribution of CENP-T within the complex, which is also capable of binding to DNA (data not shown). Thus the DNA binding activity of the CENP-T/W complex is essential for kinetochore assembly.

### Example 4

### The N-Terminus of CENP-T is Required for Kinetochore Assembly

The DNA binding activity of CENP-T resides in a histone-fold-like C-terminal region of the protein (Hori et al., 2008). To probe the function of the CENP-T N-terminus, we analyzed its role in vivo by RNAi complementation. Depletion of endogenous CENP-T by RNAi caused a mitotic arrest with unaligned chromosomes in HeLa cells. This depletion could be rescued by expression of RNAi resistant GFP-CENP-T (FIG. 2D). However, expression of GFP-CENP-T ΔN-terminus (lacking amino acids 1-287) could not rescue the phenotype, despite its localization to centromeres at comparable levels to the wild-type protein (FIG. 2D). This indicates that the N-terminus of the CENP-T is essential for accurate chromosome segregation. Recombinant CENP-T ΔN-terminus retained normal CENP-W binding properties (FIG. 2A), as well as DNA binding activity (FIG. 2B), suggesting the mitotic defects in cells expressing CENP-T ΔN-terminus are due to a distinct role for the N-terminus in kinetochore function. Consistent with this, the localization of CENP-H is lost after CENP-T depletion, and could be restored by expression of wild type CENP-T, but not CENP-T ΔN-terminus (data not shown).

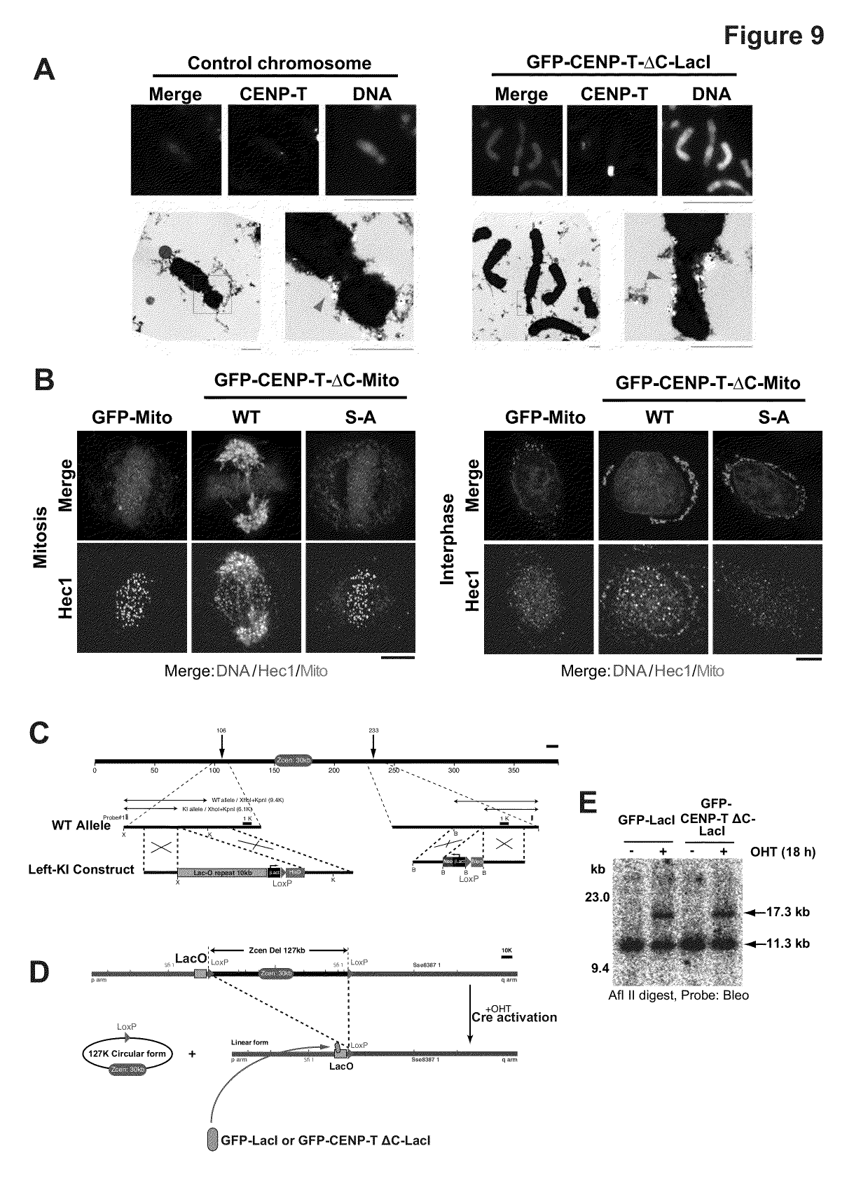

Size exclusion chromatography of recombinant CENP-T/W complex indicated that it has an elongated structure, with a Stokes radius of ˜5.5 nm (FIG. 21). Sucrose gradients indicated the complex does not exist as an oligomer (FIG. 9), and removal of the N-terminal 288 amino acids dramatically shifted the elution profile of the complex (FIGS. 2A and I), suggesting that the N-terminus of CENP-T has an extended structure. Analysis of the N-terminus of CENP-T in vivo indicated that is essential for kinetochore assembly and mitotic progression in both human cells and chicken DT40 cells. GFP-CENP-T ΔN-terminus (lacking amino acids 1-287 in human cells, or 1-100 in chicken DT40 cells), failed to rescue the mitotic arrest and delocalization of Hec1/Ndc80 from kinetochores following CENP-T depletion (FIG. 2D-H), further suggesting that the N-terminus of CENP-T functions in recruiting downstream kinetochore components.

### Example 5

### Artificial Targeting of CENP-C and CENP-T to Chromatin is Sufficient for Core Kinetochore Assembly

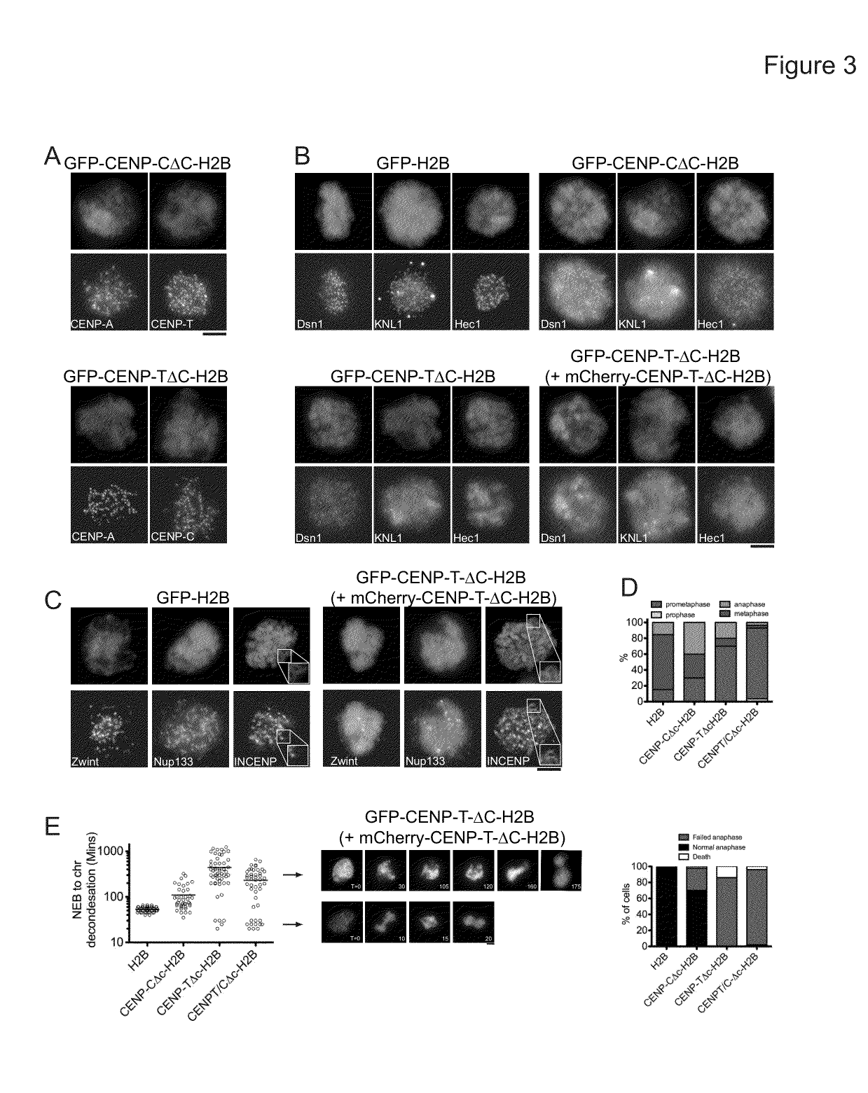

Above, we demonstrated that ectopic localization of CENP-A is not sufficient for the recruitment of the majority of kinetochore proteins to chromatin, suggesting additional factors play a role in kinetochore assembly in human cells. Both CENP-T/W and CENP-C are essential for kinetochore assembly. In contrast to CENP-A, over-expression of CENP-T/W or CENP-C did not result in incorporation at non-centromeric foci (data not shown). We therefore developed an assay to ectopically localize CENP-T and CENP-C to chromosome arms, paralleling the localization observed following over-expression of CENP-A, but bypassing the requirement for CENP-A and the intrinsic DNA binding activities of the CENP-T/W complex and CENP-C. Like CENP-T, the reported DNA binding activity of CENP-C resides in the C-terminal portion of the protein (Sugimoto et al., 1997; Yang et al., 1996). We therefore tested whether the N-terminal regions of CENP-C and CENP-T were sufficient to form a platform for kinetochore assembly. To do this, we replaced the DNA-binding domains of both CENP-T and CENP-C with histone H2B. Transient expression of the GFP-CENP-C-ΔC-H2B and GFP-CENP-T-ΔC-H2B fusion proteins in HeLa cells resulted in their incorporation throughout the chromatin (FIG. 3A).

We first investigated the localization of chromatin proximal kinetochore proteins in the presence of the CENP-T and CENP-C-H2B fusions. In the presence of GFP-CENP-C-ΔC-H2B (containing amino acids 1-235 of CENP-C), CENP-A and CENP-T localization remained restricted to centromeric foci (FIG. 3A). Similarly, in the presence of GFP-CENP-T-ΔC-H2B (containing amino acids 1-242 of CENP-T), CENP-A and CENP-C remained at centromeres, indicating that neither CENP-A nor the reciprocal DNA binding kinetochore component was recruited to chromatin by CENP-T or CENP-C mis-targeting.

We next investigated the localization of the KMN network in the presence of ectopically localized CENP-T or CENP-C. Expression of GFP-H2B as a control did not alter the exclusively centromeric localization of the KNL1, Mis12 complex subunit Dsn1, or the Ndc80 complex subunit Ndc80/Hec1 (FIG. 3B). In contrast, expression of CENP-T-ΔC-H2B or CENP-C-ΔC-H2B led to the ectopic localization of all three KMN network components to chromosome arms (FIG. 3B). However, significant differences were observed in the relative abundance of Ndc80/Hec1, Dsn1 and KNL1 on chromosome arms following expression of the individual fusion proteins. Expression of CENP-T-ΔC-H2B led to significant mis-localization of Dsn1 and KNL1, but more moderate mis-localization of Ndc80/Hec1, while expression of CENP-T-ΔC-H2B led to significant mis-localization of KNL1 and Ndc80/Hec1, but more moderate mis-localization of Dsn1. Simultaneous expression of mCherry-CENP-T-ΔC-H2B and GFP-CENP-C-ΔC-H2B led to dramatic mis-localization of all three proteins to chromosome arms during mitosis, obscuring the localization to centromeric foci. These results are consistent with our depletion analysis, which suggested a potential bias in the requirement for CENP-C and CENP-T in the recruitment of KMN network components.

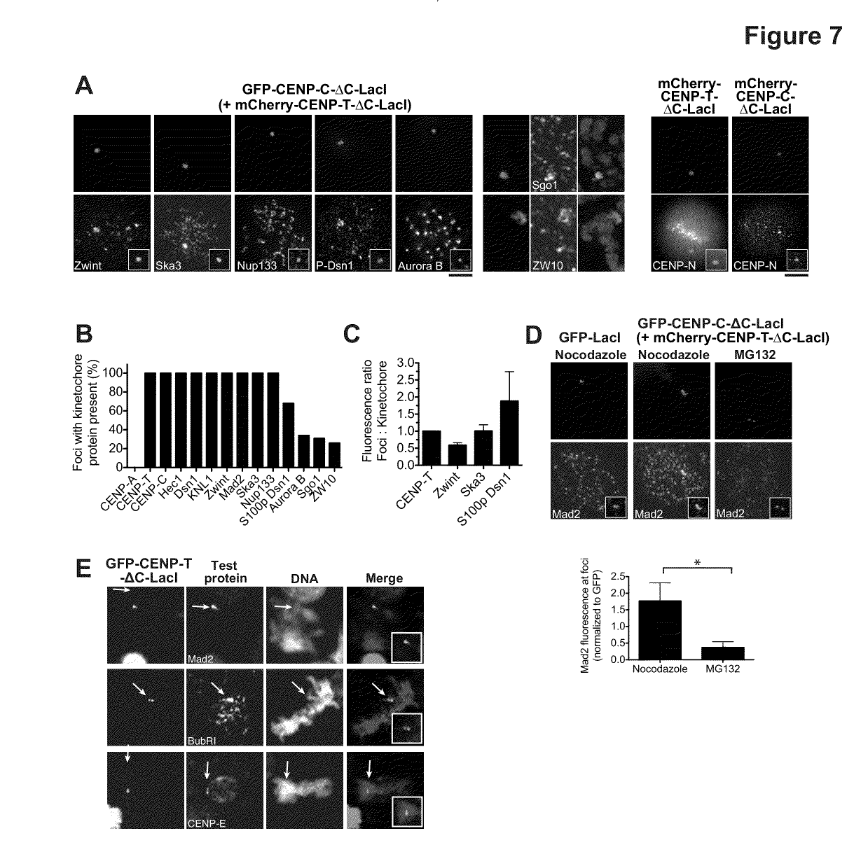

In addition to the core DNA and microtubule binding kinetochore proteins, we also investigated the localization of a panel of other kinetochore proteins in this assay. Targeting of CENP-T or CENP-C to chromosome arms resulted in the ectopic targeting of including the KMN network binding protein Zwint, the kinetochore nucleoporin Nup107-160 complex subunit Nup133, and components of the Chromosome Passenger Complex (CPC), but not Bub1, BubR1, MCAK, CENP-E, or Ska1 (FIG. 3C, FIG. 7, and data not shown). The microtubule binding Ska1 complex subunit Ska3 and CENP-N were also observed (FIG. 7A). The majority of these proteins were observed at 100% of foci (FIG. 7B), and Ska3 targeted to ectopic foci with a stoichiometry similar to that at endogenous kinetochores. We also obtained similar results using H2B fusions (FIG. 3C).

We examined CENP-T/C foci for the presence of kinetochore regulatory proteins. We found that Aurora B kinase and INCENP, components of the Chromosomal Passenger Complex (CPC), localized to both ectopic foci (FIG. 7A) and chromosome arms in the H2B fusions (FIG. 3C). However, we note that Aurora B signals were only observed at 34% of mitotic LacO foci (FIG. 7B). Consistent with the recruitment of Aurora B, Dsn1 present at ectopic LacO foci was phosphorylated at an established Aurora B phosphorylation site based on phospho-antibody localization (FIG. 7C; (Welburn et al., 2010). We also observed the MEI-S332/Shugoshin-family protein Sgo1 at ectopic CENP-T/C foci in 31% of cells.

We also analyzed the ectopic kinetochore-like foci for components of the spindle assembly checkpoint (SAC), and observed the recruitment of Mad2 and ZW10 to ectopic CENP-T/C foci in human cells (FIGS. 7A and 7D), and Mad2, BubR1, CENP-E, and ZW10 in chicken DT40 cells (FIG. 7E and data not shown). We observed a dramatic increase in the recruitment of Mad2 to the ectopic CENP-T/C foci in absence of microtubules (FIG. 7D), similar to what is observed at endogenous kinetochores. The targeting of these diverse regulatory kinetochore proteins, and the microtubule-sensitive recruitment of Mad2, is consistent with functional kinetochore-like structures at these sites.

### Example 6

### Mis-Targeting of CENP-C and CENP-T Prevents Accurate Chromosome Segregation

The mis-localization of multiple kinetochore proteins to non-centromeric sites using the CENP-T and CENP-C H2B fusion proteins led us to investigate the fidelity of chromosome segregation in these cells. Analysis of fixed cells indicated expression of CENP-C-ΔC-H2B or CENP-T-ΔC-H2B led to a higher proportion of cells with unaligned chromosomes than expression of GFP-H2B, suggesting a defect in chromosome alignment (FIG. 2D). Live cell imaging revealed that cells expressing the CENP-T or CENP-C-H2B fusion proteins failed to align chromosomes at a metaphase plate, were substantially delayed in mitosis, and failed to complete a normal anaphase (FIG. 2E). It was noted that chromosomes in the arrested cells moved rapidly, consistent with attachment to microtubules. This contrasts to cells treated with nocodazole or depleted for the Ndc80 complex subunit Nuf2, where chromosome movement is absent due to a lack of microtubule attachments (data not shown). Expression of CENP-T-ΔC-H2B alone or in combination with CENP-C-ΔC-H2B caused a more severe mitotic defect than individual CENP-C-ΔC-H2B expression, with cells arresting in mitosis for over 500 minutes before exiting aberrantly (FIG. 2E). Intriguingly, approximately 25% of mitotic cells co-expressing both fusion proteins exited mitosis very rapidly, with a mean mitotic duration of just 23 minutes, compared to 53 minutes in control GFP-H2B expressing cells. Such cells appeared to exit mitosis and undergo chromatin decondensation prior to full chromosome alignment or anaphase, a phenotype suggestive of a defect in Spindle Assembly Checkpoint (SAC) signaling. Taken together, these data demonstrate that CENP-T and CENP-C are sufficient for the recruitment of kinetochore proteins in the absence of CENP-A or centromeric chromatin, and this ectopic recruitment has functional consequences preventing accurate chromosome segregation.

### Example 7

### The N-Terminal Regions of CENP-T and CENP-C Interact Directly with Components of the KMN Network

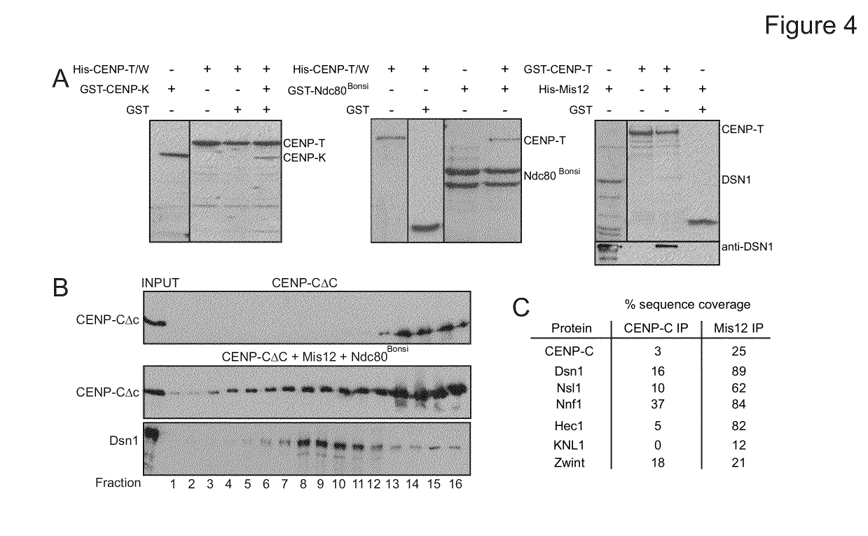

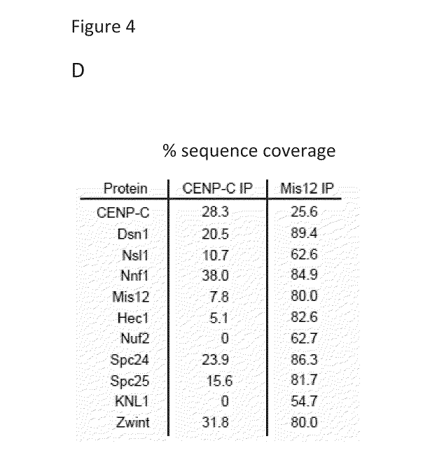

The observation that KMN network components could be recruited to chromatin by the N-terminal region of CENP-T and CENP-C led us to analyze the capacity for direct binding interactions between these proteins. To test this, we carried out binding assays with recombinant CENP-T/W complex and CENP-C-ΔC (amino acids 1-235). In bead-based pull-down assays, CENP-T bound directly to Ndc80^{Bonsai} complex and CENP-K, and weakly to the Mis12 complex (FIG. 4A). In addition, altered migration in gel filtration chromatography indicated direct binding between preassembled Ndc80^{Bonsai}/Mis12 (MN) complex and CENP-C-ΔC (FIG. 4B). Consistent with a direct interaction between CENP-C and the Mis12 complex, CENP-C isolated from HeLa cells co-purified with Dsn1, Nsl1 and Nnf1 (FIG. 4C). Together with the in vivo RNAi depletion analysis and ectopic localization experiments described above, these data strongly suggest that the N-terminal regions of CENP-T and CENP-C can each act to directly recruit the KMN network to kinetochores during mitosis.

### Example 8

### Ectopic Localization of CENP-T and CENP-C Recruits Kinetochore Proteins to Non-Centromeric Foci

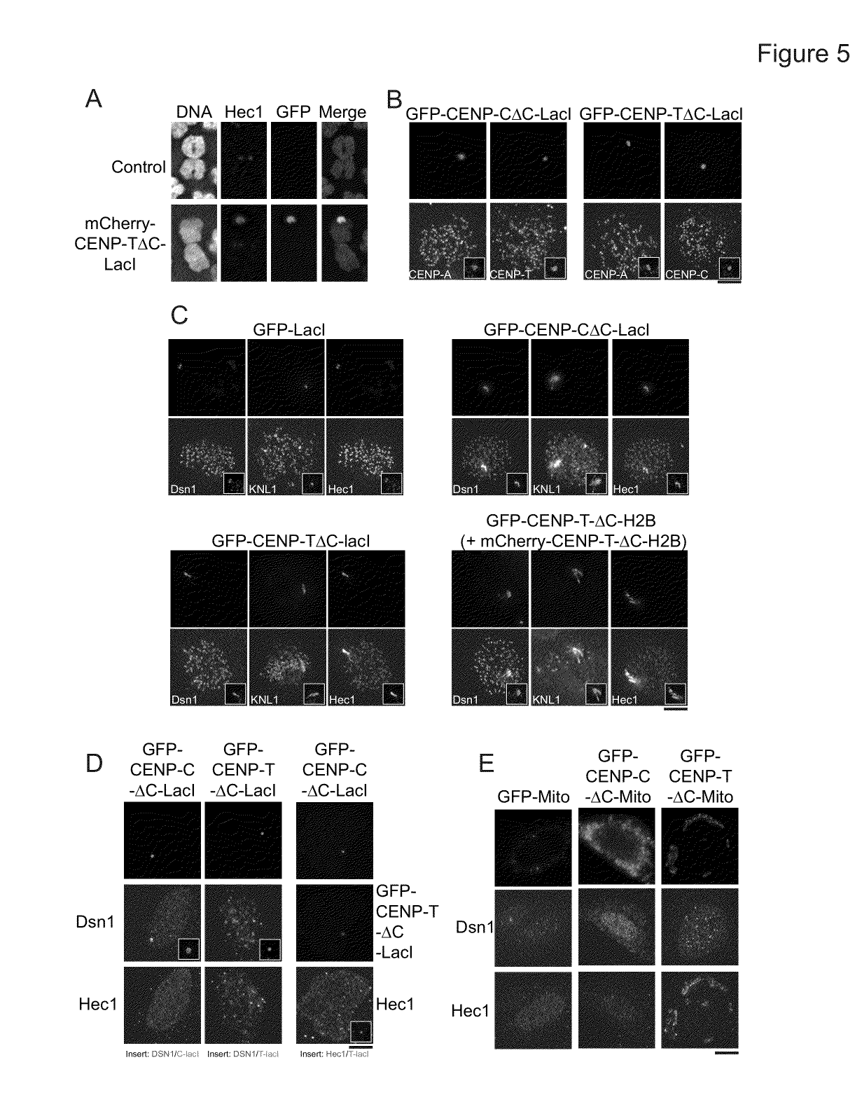

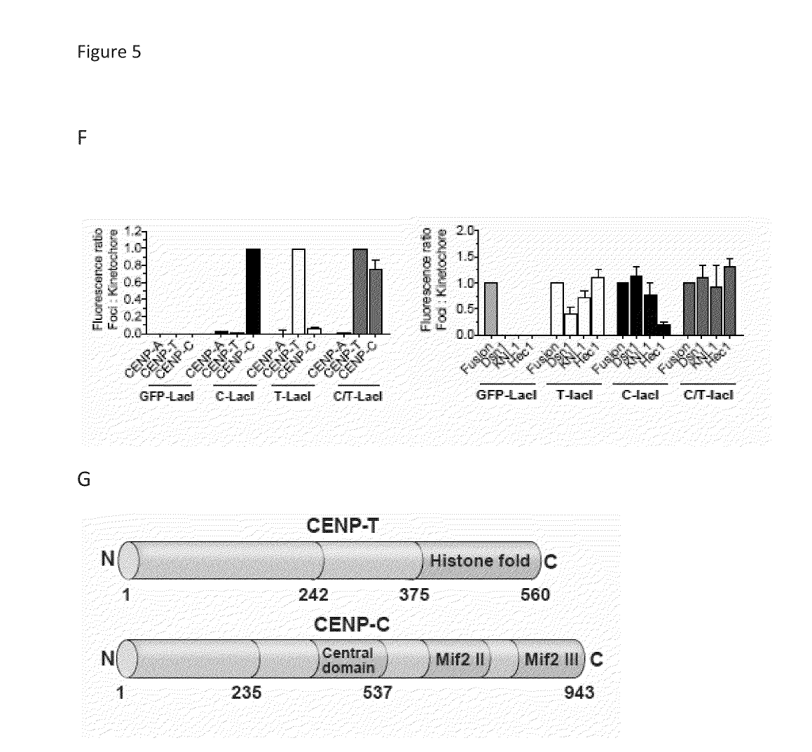

The experiments described above suggest that the CENP-T/W complex and CENP-C provide a platform for kinetochore assembly in human cells. In this case, specific targeting of these proteins to an ectopic chromosomal locus should induce formation of a kinetochore structure. To test this, we developed an assay to mis-target CENP-T and CENP-C to a single non-centromeric site in each cell in the absence of CENP-A using a Lac-operator/LacI system. GFP-LacI, GFP-CENP-C-ΔC-LacI, and GFP-CENP-T-ΔC-LacI fusion proteins were expressed in a U2OS cell line with an array of 256 Lac-operator repeats integrated into the P arm of chromosome 1 (Janicki et al., 2004). GFP-LacI could be visualized in transfected cells as a discrete GFP focus on one chromosome per cell (FIG. 5B). Similarly, GFP-CENP-C-ΔC-LacI and GFP-CENP-T-ΔC-LacI proteins could also be visualized as a single focus in each transected cell (FIG. 5A). In the presence of either GFP-CENP-C-ΔC-LacI or GFP-CENP-T-ΔC-LacI, neither CENP-A, CENP-T or CENP-C co-localized with the GFP foci (FIG. 5B), However, consistent with the analysis of H2B fusions described above, all components of the KMN network co-localized with GFP-CENP-C-ΔC-LacI and GFP-CENP-T-ΔC-LacI foci (FIG. 5C). This data indicates that ectopic localization of CENP-C or CENP-T to single foci in cells is sufficient for recruitment of the core kinetochore proteins.

### Example 9

### The Temporal Regulation of KMN Network Recruitment to Centromeres is Preserved at Ectopic Kinetochore Foci

While CENP-C and CENP-T are present at the centromere throughout the cell cycle, the majority of outer kinetochore proteins are recruited upon entry into mitosis, indicating that the assembly of the kinetochore is a highly regulated process. We used our ectopic localization strategy to probe this regulation by extending our analysis to investigate the interphase localization of kinetochore proteins. In unperturbed cells, the Mis12 complex localized to interphase centromeres in a subset of cells, while the Ndc80 complex is absent from centromeres until mitotic entry (Cheeseman et al., 2008). To determine whether the temporal regulation of this recruitment also occurs at induced ectopic foci, we monitored Mis12 complex localization to CENP-T/CENP-C-LacI foci during interphase. Dsn1 co-localized with GFP-CENP-C-ΔC-LacI foci, but not GFP-CENP-T-ΔC-LacI foci in interphase (FIG. 5D). In contrast, Ndc80/Hec1 did not co-localize with GFP-CENP-C-ΔC-LacI or GFP-CENP-T-ΔC-LacI foci in interphase, indicating that temporal regulation of this recruitment is maintained, and is not dependent on the presence of centromeric DNA or CENP-A.

To determine whether the absence of Ndc80 binding in interphase is regulated by the cell cycle, or if it is related to nuclear exclusion of Ndc80, we artificially targeted CENP-T or CENP-C to the cytosol by generating fusions with a mitochondrial outer membrane protein (GFP-CENP-T/C-ΔC-mito). Surprisingly, while GFP-CENP-C-ΔC-mito failed to recruit Dsn1 or Ndc80/Hec1 to the cytosol, Ndc80/Hec1 co-localized with GFP-CENP-T-ΔC-mito foci to interphase mitochondria (FIG. 5E). Thus, while the interaction of the Mis12 complex with CENP-C is regulated within the nucleus, the interaction of the Ndc80 complex with CENP-T is regulated by spatial separation of these proteins during interphase. Taken together, these data suggest that the temporal regulation of KMN network binding at kinetochores is not dependent on centromere localization, and occurs in part due to nuclear exclusion of kinetochore proteins during the cell cycle.

### Example 10

### Induced Kinetochore-Like Foci Function in Chromosome Segregation

Above, we demonstrate that the induced targeting of CENP-C and CENP-T to ectopic foci results in formation of kinetochore-like structures based on the recruitment of KMN network components. To evaluate the functionality of these ectopic kinetochore protein foci, we chose to test three criteria; 1) the presence of regulatory/outer kinetochore proteins, 2) interactions with microtubules, 3) the segregation behavior the ectopic foci.

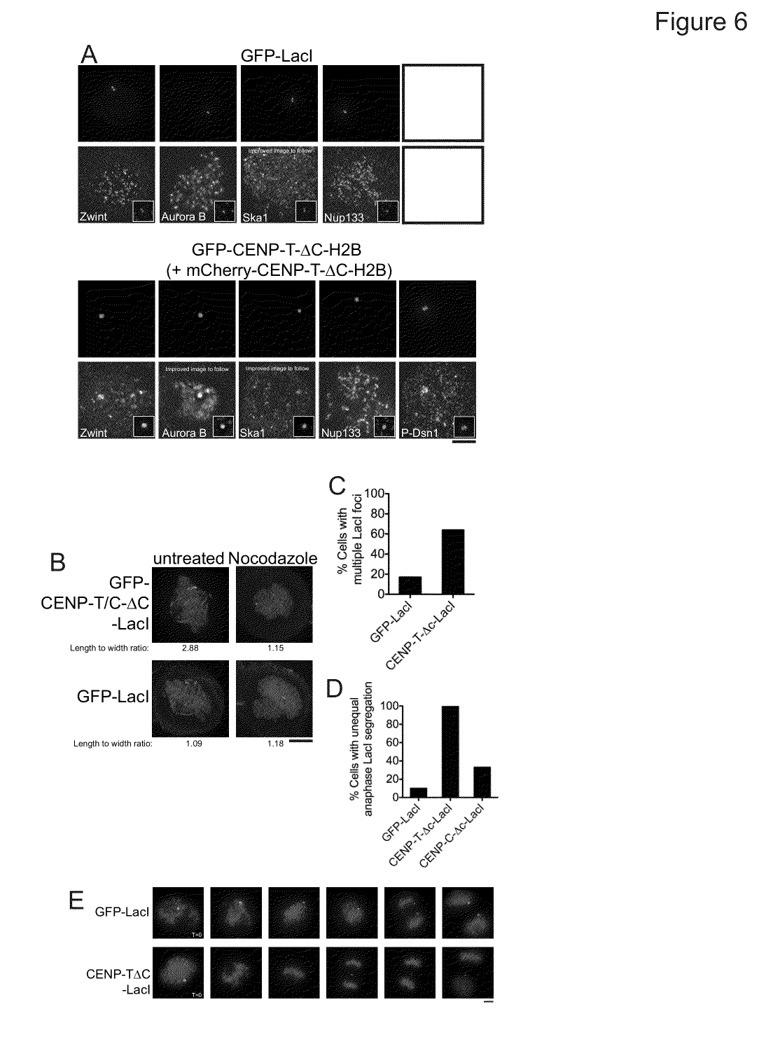

To determine if ectopic CENP-T/CENP-C foci have the potential to recruit regulatory kinetochore proteins to chromatin, we carried out a detailed immunofluorescence analysis. In addition to KMN network components, multiple kinetochore proteins also co-localized with GFP-CENP-C-ΔC-LacI and GFP-CENP-T-ΔC-LacI foci including Zwint, Nup133, Ska1, and Aurora B (FIG. 6A). We also noted that Dsn1 present at foci was phosphorylated at a known Aurora B phosphorylation site (Welburn et al., 2010), suggesting the protein was regulated in a manner similar to that at endogenous kinetochores. The presence of these additional diversely behaved proteins is consistent with a functional kinetochore-like structure at these sites.

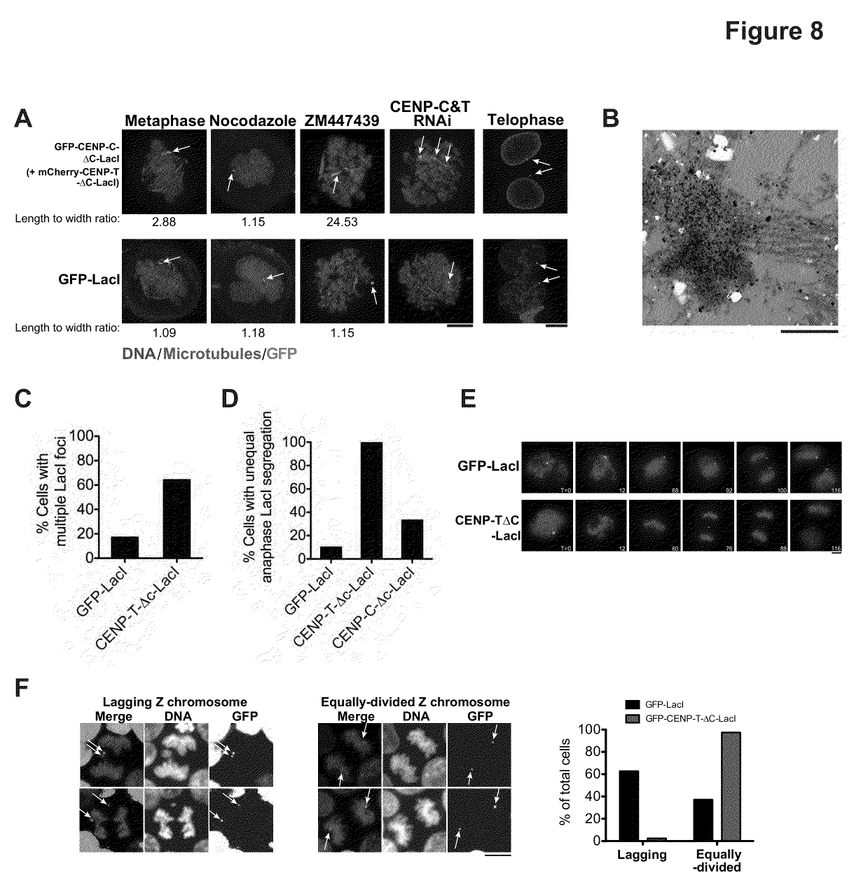

We next sought to test the interaction of these foci with spindle microtubules. Importantly, we found that the GFP-LacI foci displayed a different morphology from GFP-CENP-T-ΔC-LacI in the presence of microtubules. While GFP-LacI foci remain circular during mitosis, GFP-CENP-T-ΔC-LacI foci formed a bar-like shape, with an average length to width ratio of 2.89, suggestive of a force being applied across the region (FIG. 6B). This deformation of the LacI foci could represent microtubules binding the structure to opposite spindle poles. In such a situation, tension across the region would result in a change in the shape of the chromatin. Consistent with microtubule interactions with the foci, the deformed shape of the GFP-CENP-T-ΔC-LacI focus was dependent on microtubules, as nocodazole treatment relaxed this to a circular morphology, with an average length to width ration of 1.15 (FIG. 6B). See also FIG. 8. Deformation of the GFP-CENP-T-ΔC-LacI focus was exacerbated by treatment with the Aurora B inhibitor ZM447439, which has been proposed to regulate both kinetochore-microtubule attachments (Cheeseman and Desai, 2008) and chromatinstructure (Lipp et al., 2007). In the absence of endogenous kinetochores (using RNAi-based depletion of endogenous CENP-T and CENP-C), robust interactions with microtubules were observed for ectopic CENP-T/C-LacI foci, but not GFP-LacI controls (FIG. 8A). These foci often appeared broken, indicating that interactions with microtubules are maintained and may cause damage due to merotelic attachments.

If GFP-CENP-T-ΔC-LacI foci can interact with microtubules, such chromosomes should behave as functionally di-centric chromosomes, with the possibility of both the ectopic and endogenous kinetochores interacting independently with the spindle poles. The segregation of such a di-centric chromosome should be strongly impaired. To test if microtubule interactions impeded the segregation of the lacO containing chromosome, we carried out imaging of fusion protein expressing cells. 72 hours after transfection, multiple foci could be observed in cells expressing GFP-CENP-T-ΔC-LacI, while control GFP-LacI expressing cells contained only one foci per cell in the majority cases (FIG. 6C). This suggests that segregation of the LacO containing chromosome is impaired in the presence of GFP-CENP-T-ΔC-LacI, resulting in co-segregation of sister chromatids to the same daughter cell (FIG. 6D). Stable cell lines expressing the GFP-LacI control protein could be easily generated in U2OS-LacO cells. However, while we easily generate GFP-CENP-T-ΔC-LacI stable cell lines in U2OS cells lacking the LacO array, we could not isolate stable cell lines in U2OS-LacO cells. This is consistent with a defect in chromosome segregation, and hence a loss of viability, in cells where a LacO/LacI kinetochore focus is present (data not shown). However, such cell lines could be generated if cells were grown in 1 mM IPTG to prevent binding of the LacI fusion protein to the LacO array.

Live cell imaging showed that control GFP-LacI expressing cells segregated the LacO containing chromosome correctly in 90% of cases, as judged by the presence of one GFP-LacI foci in each daughter cell after anaphase (FIG. 6D). In contrast, in cells expressing GFP-CENP-T-ΔC-LacI, segregation of the LacO containing chromosome was impeded, with the GFP-CENP-T-ΔC-LacI foci lagging behind the separating chromatin masses at anaphase and eventually being retained in only one of the two daughter cells (FIG. 6E). This behavior is consistent with that of a di-centric chromosome, where merotelic interactions can persist into anaphase.

Taken together, these data strongly suggest that CENP-T and CENP-C form the platform for outer kinetochore assembly in human cells. Based on the recruitment of diverse outer kinetochore proteins to ectopic CENP-T and CENP-C foci, evidence of microtubule interactions, and the impairment of segregation of chromosomes where foci are present, these induced kinetochore foci are at least partial functional. Thus, CENP-C and CENP-T are sufficient for the recruitment of the microtubule binding activity of the kinetochore to chromatin bypassing the requirement for CENP-A.

Electron microscopy analysis of CENP-T-LacI foci also indicated the presence of microtubule attachments at these sites (FIG. 7B) and the formation of constriction similar to that found at endogenous kinetochores (FIG. 9A). The interaction of CENP-T-based structures with microtubules does not appear to require the presence of chromosomal DNA, as when CENP-T was targeted to the mitochondrial outer membrane, mitochondria redistributed to the mitotic spindle suggesting the presence of microtubule interactions (FIG. 9B). These data further confirm that induced ectopic CENP-T foci can form interactions with microtubules.

### Example 11

### Further Evaluating the Functionality of Engineered Kinetochores

The functionality of ectopic targeting of CENP-C and CENP-T polypeptides to generate stable segregation of a piece of DNA is further evaluated using DNA introduced into human tissue culture cells and/or using a centromere replacement assay to evaluate whether the kinetochore foci generated by CENP-C/T targeting are fully functional and sufficient for accurate segregation in the absence of an endogenous centromere. For the latter assay, cell lines are generated in DT40 chicken cells, where the endogenous centromere of the Z chromosome is removed and instead the chromosome relies on an ectopic LacI-CENP-C/CENP-T focus for segregation. Survival of such a cell line confirms that the ectopic system is capable of sustaining accurate segregation, and, therefore, could be used to segregate a HAC.

### Example 12

### Induced CENP-T-ΔC-LacI Foci can Direct Chromosome Segregation in the Absence of an Endogenous Kinetochore

As outlined in Example 11, to assess the function of an ectopic kinetochore focus in the absence of an endogenous kinetochore, a centromere replacement system in chicken DT40 cells was constructed. The chicken Z chromosome was modified to insert LacO repeats ˜50 kb from the endogenous Z centromere (FIGS. 9C and D), and loxP sites were placed flanking the centromere region. Activation of Cre recombinase resulted in excision of the endogenous centromere (Shang et al., 2010) leaving only the LacO repeats (FIG. 9E). The segregation behavior of the Z chromosome in the first division after centromere removal was assessed. Over 60% of cells expressing GFP-LacI displayed lagging chromosomes following centromere excision (FIG. 8F). In contrast, in cells with GFP-CENP-T-ΔC-LacI foci, lagging centromeres were observed in less than 3% of cells. Although expression of GFP-CENP-T-ΔC-LacI does not rescue the long-term viability of these cells (most likely for technical reasons due to a lower frequency of foci formation), these data confirm that CENP-T-induced ectopic foci are sufficient to drive correct segregation of the chromosome and are able to at least partially replace endogenous kinetochore function.

Gascoigne K E, et al, Induced ectopic kinetochore assembly bypasses the requirement for CENP-A nucleosomes. Cell 145(3):410-22 (2011) is incorporated herein by reference, including all text, figures, supplemental figures, tables, movies, and references cited therein.

### Experimental Procedures Used in the Examples

Cell Culture and Transfection

Cell lines were maintained in Dulbecco's modified Eagle's medium (DMEM) supplemented with 10% fetal bovine serum (FBS), penicillin/streptomycin, and L-glutamine (Invitrogen) at 37° C. in a humidified atmosphere with 5% CO₂. A U2OS lacO cell line was maintained in 200 μg/ml Hygromycin B (a generous gift from S Janicki). The BBB neo-centromere cell line was cultured as described previously (Alonso et al., 2007). Stable clonal cells lines expressing GFP^{LAP} fusions were generated in HeLa cells as described previously (Cheeseman et al., 2004). CENP-T and CENP-W ecoli codon optimized cDNAs were used in all experiments. GFP and mCherry fusion protein plasmids where generated in either pEGFP or pBABE backbones. Transient transfections were carried out using Effectine (Quigen) according to manufacturers instructions. Cells were observed 28 hours after transfection. Pools of siRNAs against CENP-W (UUACUCGCACAAGCGUUUGUU, UUUCGCUUGAAGACUCGCUUUN, UAAUCGAUGAACAAACAGUUUM, UUCCGCUUUAUCUGCUUCCUU), CENP-T (CAAGAGAGCAGUUGCGGCA, GACGAUAGCCAGAGGGCGU, AAGUAGAGCCCUUACACGA, CGGAGAGCCCUGCUUGAAA) and CENP-C (GCGAAUAGAUUAUCAAGGAUU, GAACAGAAUCCAGCACAAAUU, CGAAGUUGAUAGAGGAUGAUU, UCAGGAGGAUUCGUGAUUAUU), and a non-targeting control were obtained from Darmacon. RNAi experiments were conducted as described previously (Kline et al., 2006).

Immunofluorescence and Microscopy

Immunofluorescence in human cells was conducted as described previously (Kline et al., 2006). Kinetochore proteins were visualized using; mouse anti-CENP-A (3-19 Abcam, Cambridge, Mass.) was used 1:1000, was used 1:1000, rabbit anti-hKNL1 and anti-Dsn1 (Kline et al., 2006) was used 1:1000, mouse anti-HEC1 (9G3; Abcam, Cambridge, Mass.) was used at 1:1000, rabbit anti-S100p-Dsn1 (Welburn et al., 2010) was used 1:1000, rabbit anti-Nup133 (a generous gift from Douglass Forbes) was used 1:200, rabbit anti-INCENP (Abcam) was used 1:1000, mouse anti-AIM1 (BD transduction Labs) was used 1:300, and rabbit anti-Ska1 (Welburn et al., 2009) was used 1:1000. Affinity purified rabbit polyclonal antibodies was generated against full length human CENP-T, CENP-H, Zwint and CENP-C N-terminus, as described previously (Desai et al., 2003), and used 1:1000. Human anti-centromere antibodies (ACA; Antibodies, Inc., Davis, Calif.) were used at 1:100. For immunofluorescence against microtubules, DM1α (Sigma) was used at 1:1000. Cy2, Cy3, and Cy5-conjugated secondary antibodies (Jackson Laboratories) were used at 1:100. DNA was visualized using 10 μg/ml Hoechst. Where indicated, cells were incubated for 15 hours with nocodazole: 0.2 μg/ml. Metaphase spreads were performed as described in (Kiyomitsu et al., 2010), FISH was performed on the neo-centromere cell line as described previously (Warburton et al., 2000). See Table 2 for further information regarding antibodies used to visualize kinetochore proteins.

TABLE 2
Antibodies used to visualize kinetochore proteins

| Protein (organism) | Antibody | Source |
|---|---|---|
| CENP-A (human) | Mouse anti-CENP-A | Abcam |
| | (3-19) | |
| KNL1 (human) | Rabbit anti-KNI1 | (Kline et al., 2006) |
| Dsn1 (human) | Rabbit anti-Dsn1 | (Kline et al., 2006) |
| HEC1/Ndc80 (human) | Anti-HEC1 (9G3) | Abcam |
| phospho-Dsn1 | Rabbit anti-phospho- | (Welburn et |
| (human) | S100-Dsn1 | al., 2010). |
| Nup133 (human) | Rabbit anti-Nup133 | Gift from Douglass |
| | | Forbes (UCSD) |
| INCENP (human) | Rabbit anti-INCENP | Abcam |
| Aurora B (human) | Mouse anti-AIM1 | BD transduction labs |
| ZW10 (human) | Rabbit anti-ZW10 | Abcam |
| CENP-E (human) | Mouse anti-CENP-E | Abcam |
| Bub1 (human) | Mouse anti-Bub1 | Abcam |
| BubR1 (human) | Mouse anti-BubR1 | Abcam |
| Mad2 (human) | Rabbit anti-Mad2 | Gift from Geert Kops |
| | | (University Medical |
| | | Center Utrecht |
| MCAK (human) | Rabbit anti-MCAK | Abcam |
| Ska3 (human) | Rabbit anti-Ska3 | (Welburn et |
| | | al,, 2009) |
| Human centromere | Human anti-centromere | Antibodies Inc. |
| proteins | antibodies (ACA) | |
| Sgo1 | Rabbit anti-Shugoshin | Abcam |
| CENP-T (human) | Rabbit anti-CENP-T | This study |
| CENP-H (human) | Rabbit anti-CENP-H | This study |
| Zwint (human) | Rabbit anti-Zwint | This study |
| CENP-C (human) | Rabbit anti-CENP-C N | This study |
| | terminus | |
| Pnospho-CENP-T | Rabbit anti-phospho- | This study |
| (human) | S47 CENP-T | |
| | (amino acids 38-56) | |
| Tubulin | Mouse anti-tubulin | Sigma |
| | (DM1α) | |
| CENP-H (chicken) | Rabbit anti-CENP-H | (Fukagawa et |
| | | al., 2001) |
| Mis12 (chicken) | Rabbit anti-Mis12 | (Kline et al, 2006) |
| Ndc80 (chicken) | Rabbit anti-Ndc80 | (Hori et al., 2003) |
| Nuf2 (chicken) | Rabbit anti-Nuf2 | (Hori et al., 2003) |
| Mad2 | Rabbit anti-Mad2 | (Kwon et al., 2007) |
| BubR1 | Rabbit anti-BubR1 | (Kwon et al., 2007) |
| CENP-E | Rabbit anti-CENP-E | (Kwon et al., 2007) |

To quantitate fluorescent intensity, individual kinetochores were selected from projections (selected based on co-localization with a separate kinetochore marker) and the integrated intensity was determined after subtracting the background fluorescence measured from adjacent regions of the cell using Metamorph. Fluorescence levels at kinetochores were normalized with respect to control cells. At least 5-10 cells were examined for each kinetochore protein.

Images were acquired on a DeltaVision Core deconvolution microscope (Applied Precision) equipped with a CoolSnap HQ2 CCD camera. 30 to 40 Z-sections were acquired at 0.2 μm steps using a 100×, 1.3 NA Olympus U-PlanApo objective with 1×1 binning. Images were deconvolved using the DeltaVision software. For time-lapse imaging live cells expressing GFP and/or mCherry fusion proteins were maintained in Co₂ independent media (Invitrogen) at 37° C., and imaged every 4 minutes. 8 Z-sections were acquired at 0.8 μm steps using a 40×NA Olympus UApo/340 objective with 1×1 binning.

Protein Purification and Biochemical Assays

For the expression and purification of the recombinant proteins, 6×His-CENP-T (full length or amino acids 288-561) and untagged CENP-W were expressed together from pST39. 6×his-Mis12 complex was expressed as described previously (Kline et al., 2006). GST-CENP-C N-terminus (amino acids 1-235), GST-CENP-T, and GST-CENP-K were expressed from pGEX-6P-1. Ndc80^{bonsai} was expressed as described previously (Ciferri et al., 2008). Proteins were purified using Glutathione agarose (Sigma) or Ni-NTA Agarose (Qiagen) according to the manufacturer's guidelines and then desalted into 25 mM Hepes pH 7.5, 200 mM NaCl, 1 mM EDTA, 10 mM Beta mercaptoethanol. Proteins were fractionated using a Superose 6 or superdex 200 size exclusion column. For In vitro binding assays, proteins were bound to Glutathione agarose or Ni-NTA Agarose, incubated with additional proteins, washed 3 times with 1×PBS, 250 mM Nacl, and 0.1% Tween 10 mM Beta mercaptoethanol, and resuspended in SDS-PAGE sample buffer.

GFP^{LAP} tagged Mis12 complex and endogenous CENP-C were isolated from HeLa cells as described previously (Cheeseman, 2005). CENP-C was isolated using rabbit anti-CENP-C antibody. Purified proteins were identified by mass spectrometry using an LTQ XL Ion trap mass spectrometer (Thermo) using MudPIT and SEQUEST software as described previously (Washburn et al., 2001).

DNA Binding Analysis

In vitro DNA binding was assessed by Electro Mobility Shift Assay (EMSA). 500 ng of the indicated protein was incubated for 10 minutes at room temperature in binding buffer (10 mM Tris pH 7.9, 150 mM KCl, 1 mM EDTA, 4 mM DTT. 50 ng of a 20 bp biotinylated double stranded DNA probe CCTTTGAGGCCTTCGTTGGA corresponding to bp 21-40 of the alpha satellite DNA sequence was then added, and incubated for a further 20 minutes. Samples were then run on a 4% non denaturing arylamide gel, semi-dry transferred to nitrocellulose membrane, and blots processed using the Chemiluminescent nucleic acid detection system (Thermo) according to manufacturers instructions. Biotinylated DNA probe with detected with HRP-coupled streptavidin (Thermo).

DNA binding preferences of the CENP-T/W complex were investigated in vitro by Protein Binding Microarray (PBM) analysis. CENP-T/W complex was incubated with microarrays containing all possible nucleotide 10 mers, and processed as described previously (Berger and Bulyk, 2009). Protein was detected using rabbit anti-CENP-T antibodies and anti-rabbit alexa-fluoro488 secondary antibodies.

Electron Microscopy

U2OS cells were treated with 500 ng/ml nocodazole or 10 μM MG132 for 2 h to prepare mitotic cells. Mitotic cells were cyto-centrifugated onto a glass slide, permeabilized by 1% Triton, and fixed in 3% paraformaldehyde/1.5% glutaraldehyde for 15 min. Samples were rinsed in 0.5% BSA/0.1% triton in PBS, incubated for 1 h at 37° C. with primary antibodies (anti-CENP-T or anti-Heel (9G3)), washed three times in 0.5% BSA/0.1% triton in PBS, and incubated with FITC-conjugated secondary antibodies and a 1.4 nm gold-labeled secondary antibodies (Nanoprobes), simultaneously for 2 h at 37° C. Samples were observed by fluorescence microscopy to identify CENP-T-Lac-1-GFP containing foci. The position of the chromosome with CENP-T-Lac-1-GFP was marked and samples were fixed with 2.5% glutaraldehyde/3% PFA in 0.1 M cacodylate buffer, pH 7.2, at 4° C. for 20 h. Samples were silver enhanced using a HQ-silver kit (Nanoprobes) according to the manufacture's protocol. Post-fixation performed in 0.5% OsO₄ on ice. The cells were dehydrated in ethanol and then infiltrated with Epon812. Polymerization was performed at 60° C. for 48 h. Serial sections were cut with an ultramicrotome equipped with a diamond knife (170 nm). Samples were stained by uranylacetate and lead citrate and imaged at room temperature using a JEM1010 TEM (JEOL) at 100 kV.

Chicken DT40 Experiments

DT40 cells were cultured and transfected as described previously (Hori et al., 2008). Mutant or full length CENP-C and CENP-T constructs under control of tetracycline repressive promoter were transfected into DT40 cells with a tet-repressible transactivator in the presence of tetracycline.

Immunofluorescent staining of chicken cells was performed as described previously (Fukagawa et al., 2001), using antibodies described in Table. Si. Images were collected with a cooled EM CCD camera (QuantEM, Roper Scientific Japan) mounted on an Olympus IX71 inverted microscope with a 100× objective lens together with a filter wheel and a DSU confocal unit. ˜20 Z-sections were acquired at 0.3 μm steps.

DT40 cells in which the centromere of chromosome Z can be conditionally removed were used for in centromere replacement assays (Shang et al., 2010). A LacO repeat was inserted at a ˜50 kb region adjacent to the centromere by homologous recombination. GFP-CENP-TΔC-LacI or GFP-LacI constructs were introduced into these cells. Using a Mer-Cre-Mer construct integrated in these cells, Cre recombinase was activated upon hydroxytamoxifen (OHT) addition. Removal of the centromere was confirmed by Southern hybridization (FIG. 9). Cells with lagging chromosomes during anaphase were counted 18 h after addition of OHT.

### REFERENCES

Alonso, A., Fritz, B., Hasson, D., Abrusan, G., Cheung, F., Yoda, K., Radlwimmer, B., Ladurner, A. G., and Warburton, P. E. (2007). Co-localization of CENP-C and CENP-H to discontinuous domains of CENP-A chromatin at human neocentromeres. Genome Biol 8, R148.
Amano M, Suzuki A, Hori T, Backer C, Okawa K, Cheeseman 1M, Fukagawa T. (2009). The CENP-S complex is essential for the stable assembly of outer kinetochore structure. J. Cell Biol., 186(2): 173-82.
Amor, D. J., and Choo, K. H. A. (2002). Neocentromeres: role in human disease, evolution, and centromere study. Am J Hum Genet. 71, 695-714.
Bassett, E. A., Wood, S., Salimian, K. J., Ajith, S., Foltz, D. R., and Black, B. E. (2010). Epigenetic centromere specification directs autot B accumulation but is insufficient to efficiently correct mitotic errors. J Cell Biol, In press.
Black, B. E., Jansen, L. E., Maddox, P. S., Foltz, D. R., Desai, A. B., Shah, J. V., and Cleveland, D. W. (2007). Centromere identity maintained by nucleosomes assembled with histone H3 containing the CENP-A targeting domain. Mol Cell 25, 309-322.
Carroll, C. W., Milks, K. J., and Straight, A. F. (2010). Dual recognition of CENP-A nucleosomes is required for centromere assembly. J Cell Biol 189, 1143-1155.
Carroll, C. W., Silva, M. C., Godek, K. M., Jansen, L. E., and Straight, A. F. (2009). Centromere assembly requires the direct recognition of CENP-A nucleosomes by CENP-N. Nat Cell Biol 11, 896-902.
Cheeseman, I. M. (2005). A Combined Approach for the Localization and Tandem Affinity Purification of Protein Complexes from Metazoans. Science's STKE 2005, p11-p11. Cheeseman, I. M., Chappie, J. S., Wilson-Kubalek, E. M., and Desai, A. (2006). The conserved KMN network constitutes the core microtubule-binding site of the kinetochore. Cell 127, 983-997.
Cheeseman, I. M., and Desai, A. (2008). Molecular architecture of the kinetochore-microtubule interface. Nat Rev Mol Cell Biol 9, 33-46.
Cheeseman, I. M., Hori, T., Fukagawa, T., and Desai, A. (2008). KNL1 and the CENP-H/1/K complex coordinately direct kinetochore assembly in vertebrates. Mol Biol Cell 19, 587-594.
Cheeseman, I. M., Niessen, S., Anderson, S., Hyndman, F., Yates, J. R., 3rd, Oegema, K., and Desai, A. (2004). A conserved protein network controls assembly of the outer kinetochore and its ability to sustain tension. Genes Dev 18, 2255-2268.
Ciferri, C., Pasqualato, S., Screpanti, E., Varetti, G., Santaguida, S., Dos Reis, G., Maiolica, A., Polka, J., De Luca, J. G., De Wulf, P., et al. (2008). Implications for kinetochore-microtubule attachment from the structure of an engineered Ndc80 complex. Cell 133, 427-439.
Dalal, Y., and Bui, M. (2010). Down the rabbit hole of centromere assembly and dynamics. Curr Opin Cell Biol.
Desai, A., Rybina, S., Muller-Reichert, T., Shevchenko, A., Hyman, A., and Oegema, K. (2003). KNL-1 directs assembly of the microtubule-binding interface of the kinetochore in C. elegans. Genes Dev 17, 2421-2435.
Dunleavy, E. M., Roche, D., Tagami, H., Lacoste, N., Ray-Gallet, D., Nakamura, Y., Daigo, Y., Nakatani, Y., and Almouzni-Pettinotti, G. (2009). HJURP Is a Cell-Cycle-Dependent Maintenance and Deposition Factor of CENP-A at Centromeres. Cell 137, 485-497.
Foltz, D. R., Jansen, L. E. T., Bailey, A. O., Iii, J. R. Y., Bassett, E. A., Wood, S., Black, B. E., and Cleveland, D. W. (2009). Centromere-Specific Assembly of CENP-A Nucleosomes Is Mediated by HJURP. Cell 137, 472-484.
Foltz, D. R., Jansen, L. E. T., Black, B. E., Bailey, A. O., Yates, J. R., and Cleveland, D. W. (2006). The human CENP-A centromeric nucleosome-associated complex. Nat Cell Biol 8, 458-469.
Fujita, Y., Hayashi, T., Kiyomitsu, T., Toyoda, Y., Kokubu, A., Obuse, C., and Yanagida, M. (2007). Priming of centromere for CENP-A recruitment by human hMis18alpha, hMis18beta, and M18BP1. Dev Cell 12, 17-30.
Harrington, J. J., Van Bokkelen, G., Mays, R. W., Gustashaw, K., and Willard, H. F. (1997). Formation of de novo centromeres and construction of first-generation human artificial microchromosomes. Nat Genet. 15, 345-355.
Hayashi, T., Fujita, Y., Iwasaki, O., Adachi, Y., Takahashi, K., and Yanagida, M. (2004). Mis16 and Mis18 are required for CENP-A loading and histone deacetylation at centromeres. Cell 118, 715-729.
Heun, P., Erhardt, S., Blower, M. D., Weiss, S., Skora, A. D., and Karpen, G. H. (2006). Mislocalization of the Drosophila centromere-specific histone CID promotes formation of functional ectopic kinetochores. Dev Cell 10, 303-315.
Hori, T., Amano, M., Suzuki, A., Backer, C. B., Welburn, J. P., Dong, Y., Mcewen, B. F., Shang, W.-H., Suzuki, E., Okawa, K., et al. (2008). CCAN Makes Multiple Contacts with Centromeric DNA to Provide Distinct Pathways to the Outer Kinetochore. Cell 135, 1039-1052.
Howman, E. V., Fowler, K. J., Newson, A. J., Redward, S., MacDonald, A. C., Kalitsis, P., and Choo, K. H. (2000). Early disruption of centromeric chromatin organization in centromere protein A (Cenpa) null mice. Proc Natl Acad Sci USA 97, 1148-1153.
Janicki, S. M., Tsukamoto, T., Salghetti, S. E., Tansey, W. P., Sachidanandam, R., Prasanth, K. V., Ried, T., Shav-Tal, Y., Bertrand, E., Singer, R. H., and Spector, D. L. (2004). From silencing to gene expression: real-time analysis in single cells. Cell 116, 683-698.
Kalitsis, P., Fowler, K. J., Earle, E., Hill, J., and Choo, K. H. (1998). Targeted disruption of mouse centromere protein C gene leads to mitotic disarray and early embryo death. Proc Natl Acad Sci USA 95, 1136-1141.
Kiyomitsu, T., Iwasaki, O., Obuse, C., and Yanagida, M. (2010). Inner centromere formation requires hMis14, a trident kinetochore protein that specifically recruits HP 1 to human chromosomes. The Journal of Cell Biology, 1-17.
Kline, S. L., Cheeseman, I. M., Hori, T., Fukagawa, T., and Desai, A. (2006). The human Mis12 complex is required for kinetochore assembly and proper chromosome segregation. The Journal of Cell Biology 173, 9-17.
Maddox, P. S., Hyndman, F., Monen, J., Oegema, K., and Desai, A. (2007). Functional genomics identifies a Myb domain-containing protein family required for assembly of CENP-A chromatin. J Cell Biol 176, 757-763.
Masumoto, H., Masukata, H., Muro, Y., Nozaki, N., and Okazaki, T. (1989). A human centromere antigen (CENP-B) interacts with a short specific sequence in alphoid DNA, a human centromeric satellite. J Cell Biol 109, 1963-1973.
Oegema, K., Desai, A., Rybina, S., Kirkham, M., and Hyman, A. A. (2001). Functional analysis of kinetochore assembly in Caenorhabditis elegans. J Cell Biol 153, 1209-1226.
Ohzeki, J., Nakano, M., Okada, T., and Masumoto, H. (2002). CENP-B box is required for de novo centromere chromatin assembly on human alphoid DNA. The Journal of cell biology 159, 765-775.
Okada, M., Cheeseman, I. M., Hori, T., Okawa, K., Mcleod, I. X., Yates, J. R., Desai, A., and Fukagawa, T. (2006). The CENP-H-I complex is required for the efficient incorporation of newly synthesized CENP-A into centromeres. Nature Cell Biology 8, 446-457.
Musacchio, A. and Salmon, E. D. (2007) The spindle-assembly checkpoint in space and time, Nat. Rev. Mol. Cell. Biol. 8, 379-393.
Santaguida S, Musacchio A. (2009) The life and miracles of kinetochores. EMBO J., 28(17):2511-31.
Sugimoto, K., Kuriyama, K., Shibata, A., and Himeno, M. (1997). Characterization of internal DNA-binding and C-terminal dimerization domains of human centromere/kinetochore autoantigen CENP-C in vitro: role of DNA-binding and self-associating activities in kinetochore organization. Chromosome Res 5, 132-141.
Sugimoto, K., Yata, H., Muro, Y., and Himeno, M. (1994). Human centromere protein C (CENP-C) is a DNA-binding protein which possesses a novel DNA-binding motif. J Biochem 116, 877-881.
Van Hooser, A. A., Ouspenski, I. I., Gregson, H. C., Starr, D. A., Yen, T. J., Goldberg, M. L., Yokomori, K., Earnshaw, W. C., Sullivan, K. F., and Brinkley, B. R. (2001). Specification of kinetochore-forming chromatin by the histone H3 variant CENP-A. J Cell Sci 114, 3529-3542.
Warburton, P. E., Dolled, M., Mahmood, R., Alonso, A., L1, S., Naritomi, K., Tohma, T., Nagai, T., Hasegawa, T., Ohashi, H., et al. (2000). Molecular cytogenetic analysis of eight inversion duplications of human chromosome 13q that each contain a neocentromere. Am J Hum Genet. 66, 1794-1806.
Washburn, M. P., Wolters, D., and Yates, J. R., 3rd (2001). Large-scale analysis of the yeast proteome by multidimensional protein identification technology. Nat Biotechnol 19, 242-247.
Welburn, J. P., Vleugel, M., Liu, D., Yates, J. R., 3rd, Lampson, M. A., Fukagawa, T., and Cheeseman, I. M. (2010). Aurora B phosphorylates spatially distinct targets to differentially regulate the kinetochore-microtubule interface. Mol Cell 38, 383-392.
Welburn, J. P. I., Grishchuk, E. L., Backer, C. B., Wilson-Kubalek, E. M., Yates, J. R., and Cheeseman, I. M. (2009). The human kinetochore Ska1 complex facilitates microtubule depolymerization-coupled motility. Dev Cell 16, 374-385.
Yang, C. H., Tomkiel, J., Saitoh, H., Johnson, D. H., and Earnshaw, W. C. (1996). Identification of overlapping DNA-binding and centromere-targeting domains in the human kinetochore protein CENP-C. Mol Cell Biol 16, 3576-3586.
Fukagawa, T., Mikami, Y., Nishihashi, A., Regnier, V., Haraguchi, T., Hiraoka, Y., Sugata, N., Todokoro, K., Brown, W., and Ikemura, T. (2001). CENP-H, a constitutive centromere component, is required for centromere targeting of CENP-C in vertebrate cells. EMBO J. 20, 4603-4617.
Hori, T., Haraguchi, T., Hiraoka, Y., Kimura, H., and Fukagawa, T. (2003). Dynamic behavior of Nuf2-Hec1 complex that localizes to the centrosome and centromere and is essential for mitotic progression in vertebrate cells. J Cell Sci 116, 3347-3362.
Kwon, M.-S., Hori, T., Okada, M., and Fukagawa, T. (2007). CENP-C is involved in chromosome segregation, mitotic checkpoint function, and kinetochore assembly. Mol Biol Cell 18, 2155-2168.
Lipp, J. J., Hirota, T., Poser, I., and Peters, J. M. (2007). Aurora B controls the association of condensin I but not condensin II with mitotic chromosomes. J Cell Sci 120, 1245-1255. Nakano, M., Cardinale, S., Noskov, V. N., Gassmann, R., Vagnarelli, P., Kandels-Lewis,
S., Larionov, V., Earnshaw, W. C., and Masumoto, H. (2008). Inactivation of a human kinetochore by specific targeting of chromatin modifiers. Dev Cell 14, 507-522.

1. A modified DNA-binding constitutive centromere associated network (DBCCAN) polypeptide that comprises a heterologous DNA binding domain.

2. The modified DBCCAN polypeptide of claim 1, wherein the polypeptide does not comprise a native DNA binding domain.

3. The modified DBCCAN polypeptide of claim 1, wherein the heterologous DNA binding domain is capable of sequence-specific binding to a DNA segment not naturally present in the human genome and not to DNA present in the human genome.

4. The modified DBCCAN polypeptide of claim 1, wherein the polypeptide is a modified CENP-C polypeptide.

5. The modified DBCCAN polypeptide of claim 1, wherein the polypeptide is a modified CENP-T polypeptide.

6. The modified DBCCAN polypeptide of claim 1, wherein the heterologous DNA binding domain is capable of binding to a bacterial operator.

7. The modified DBCCAN polypeptide of claim 6, wherein the bacterial operator is the Lac operator or the Tet operator.

8. The modified DBCCAN polypeptide of claim 1, wherein the polypeptide comprises a tag or a detectable or selectable marker.

9. A nucleic acid comprising a polynucleotide that encodes the modified DBCCAN polypeptide of claim 1.

10. The nucleic acid of claim 9, wherein the polynucleotide that encodes the modified DBCCAN polypeptide is operably linked to an expression control element.

11.-25. (canceled)

26. A nucleic acid-protein structure comprising (a) the modified DBCCAN polypeptide of claim 1; and (b) a nucleic acid comprising a DNA segment cognate to the heterologous DNA binding domain, wherein the modified DBCCAN polypeptide is bound to the DNA segment.

27. (canceled)

28. The nucleic acid-protein structure of claim 26, which comprises (a) a modified CENP-C polypeptide and a modified CENP-T polypeptide; (b) a nucleic acid comprising (i) a DNA segment cognate to the heterologous DNA binding domain of the modified CENP-C polypeptide and (ii) a DNA segment cognate to the heterologous DNA binding domain of the modified CENP-T polypeptide; and wherein the modified CENP-C polypeptide and the modified CENP-T polypeptide are bound to their cognate DNA segments.

29. (canceled)

30. The nucleic acid-protein structure of claim 28, further comprising at least some components of the KNL-1/Mis12 complex/Ndc80 complex (KMN) network.

31.-47. (canceled)

48. An engineered kinetochore comprising: (a) a modified CENP-C polypeptide comprising a heterologous DNA binding domain; (b) a modified CENP-T polypeptide comprising a heterologous DNA binding domain; and (c) at least some KMN components; and, optionally, (d) at least some additional outer kinetochore proteins.

49. (canceled)

50. A nucleic acid-protein structure comprising the engineered kinetochore of claim 48 and DNA, wherein the kinetochore is assembled on the DNA.

51. The nucleic-acid protein structure of claim 50, wherein the site at which the kinetochore assembles substantially lacks CENP-A nucleosomes.

52. A chromosome comprising the nucleic-acid protein structure of claim 50.

53. The chromosome of claim 52, wherein the chromosome is an artificial chromosome.

54.-55. (canceled)

56. A eukaryotic cell comprising the chromosome of claim 52.

57. The eukaryotic cell of claim 56, wherein the cell is a vertebrate cell, which is optionally a mammalian or avian cell.

58.-76. (canceled)


SEQUENCE LISTING (13 sequences)

SEQ ID NO: 1 - RNA, 21 nt, Artificial
  siRNAs against CENP-W
uuacucgcacaagcguuuguu

SEQ ID NO: 2 - RNA, 22 nt, Artificial
  siRNAs against CENP-W
uuucgcuugaagacucgcuuun

SEQ ID NO: 3 - RNA, 22 nt, Artificial
  siRNAs against CENP-W
uaaucgaugaacaaacaguuum

SEQ ID NO: 4 - RNA, 21 nt, Artificial
  siRNAs against CENP-W
uuccgcuuuaucugcuuccuu

SEQ ID NO: 5 - RNA, 19 nt, Artificial
  siRNAs against CENP-T
caagagagcaguugcggca

SEQ ID NO: 6 - RNA, 19 nt, Artificial
  siRNAs against CENP-T
gacgauagccagagggcgu

SEQ ID NO: 7 - RNA, 19 nt, Artificial
  siRNAs against CENP-T
aaguagagcccuuacacga

SEQ ID NO: 8 - RNA, 19 nt, Artificial
  siRNAs against CENP-T
cggagagcccugcuugaaa

SEQ ID NO: 9 - RNA, 21 nt, Artificial
  siRNAs against CENP-C
gcgaauagauuaucaaggauu

SEQ ID NO: 10 - RNA, 21 nt, Artificial
  siRNAs against CENP-C
gaacagaauccagcacaaauu

SEQ ID NO: 11 - RNA, 21 nt, Artificial
  siRNAs against CENP-C
cgaaguugauagaggaugauu

SEQ ID NO: 12 - RNA, 21 nt, Artificial
  siRNAs against CENP-C
ucaggaggauucgugauuauu

SEQ ID NO: 13 - DNA, 20 bp, Artificial
  double stranded DNA probe
cctttgaggccttcgttgga